SEQUENCE LISTING (4 sequences)

SEQ ID NO: 1 - protein, 883 aa, synthetic construct
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 2 - DNA, 2652 bp, synthetic construct
  source 1..2652: mol_type = other DNA
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 3 - DNA, 4684 bp, synthetic construct
  source 1..4684: mol_type = other DNA
ttaatacgactcactataggaataaactagtattcttctggtccccacagactcagagagaacccgccaccatggataaaaagtattcaataggactagacattggcaccaattccgtgggctgggcagttattactgacgaatataaagttccgagcaaaaagttcaaagtattaggcaatacggatcgccatagcatcaaaaaaaatctgatcggtgccttgctgttcgactccggggaaaccgcggaagcgacccgtctgaagagaaccgctcgtagacgctacacccgccgcaaaaaccgtatctgctatttgcaagagatctttagcaacgagatggcgaaggttgacgactccttttttcatcgtttggaggaaagtttcctggtcgaggaggacaagaaacacgagcgtcatccgatttttggcaacatcgtcgacgaagttgcgtatcacgaaaaataccccacgatctaccatctgcgtaagaagctcgttgactccaccgataaggcggatctgcgtctgatctatctggcactggctcacatgattaaattccgtggtcactttctgatcgagggtgatctcaacccggataatagcgatgttgataagcttttcatccagctggtccagacatacaaccaattgttcgaggagaacccgattaatgcgtctggtgttgatgctaaagcgattctttctgcgcgtttatccaagtcgcgtcgcctggaaaacctgattgctcaactcccaggtgagaaaaaaaacggtctgttcggtaacttgattgcgctgagcctgggcctgacgccgaatttcaaaagcaatttcgacctggcggaagacgcgaagctgcaattaagcaaagacacatacgatgacgacctagacaacctgcttgcccaaattggcgaccagtatgctgacttgttcttagctgcaaaaaacctctccgacgcgattttgctgagcgatatcttgcgtgttaacaccgaaatcactaaagctccgctgagcgctagcatgatcaaacgctacgatgagcatcaccaggatctgaccctgctgaaggctctcgtgcagcaacagttaccagagaaatacaaggagatcttctttgatcaatctaaaaacggttatgcgggttacattgacggcggcgcgagccaagaagagttttataagttcattaagccgattcttgagaagatggatggtactgaggaactgctggtcaaacttaaccgtgaagacctgctgcgcaagcagcgtacctttgacaacggctcaattccgcatcagatccacctcggtgagctgcacgccattctgcgccgtcaagaagatttttacccgtttttgaaggataaccgtgagaaaatcgaaaagatcttgacgtttcgtatcccgtattacgtgggtccgctggcgcgtggtaatagccgttttgcgtggatgacccgcaagagcgaggaaaccatcaccccgtggaatttcgaagaggtggtggataagggcgcgtctgcccaatcctttatcgagcgcatgaccaactttgacaagaacctgccgaatgagaaggtgctgccaaagcatagcctcttgtacgagtacttcaccgtttataacgaattgaccaaagtgaagtacgttaccgaaggtatgcgtaaaccggcatttctgtctggtgaacagaagaaggccattgtggacctgctgttcaaaaccaatcggaaagttaccgttaaacagttgaaagaagattattttaaaaaaattgaatgttttgactccgtagagatctctggcgttgaggaccgctttaacgcgtccttaggtacgtaccatgatctcctgaaaattatcaaggacaaggacttcctggataatgaagagaacgaagatatcctggaagacatcgttctgacgctgaccctgttcgaagaccgtgaaatgattgaagagcgcttgaaaacgtacgcccacctgttcgatgataaagtaatgaaacaactgaagcgccgtcgctataccggttggggtcgtctgtctagaaagctcattaacggcatccgcgacaagcaaagcggcaaaaccatcctggactttcttaaatctgatggttttgcgaaccgcaacttcatgcagttgatccacgacgactctctgacgtttaaagaagatatccagaaagcgcaagttagcggtcagggtgatagcctgcacgaacacattgcgaacctggcgggtagtccagcgatcaaaaagggtattctgcagacggtcaaggttgtggatgaactggttaaagttatgggtcgacacaaaccggaaaacattgtgatcgagatggcacgtgaaaaccagaccacgcaaaaaggccagaagaattctagagaacgtatgaaacgcatcgaagagggcatcaaagagctcggctctcaaatattgaaggaacatccggttgagaacacccaattgcaaaatgagaaactgtatttgtattacctgcaaaacggtcgtgatatgtatgtggaccaggaactggatatcaatcgtctctctgactacgacgtggaccacatcgtgccgcagagtttcctgaaagatgattcgattgacaacaaagtgctgacccgtagcgataagaatagaggcaaaagcgacaacgtgcctagcgaggaagtcgtcaagaaaatgaaaaattattggcgtcagctgctgaatgccaagctgatcacccagcgtaaattcgacaacctgaccaaagccgaacgcggtggcctgtccgagctggacaaggccggttttattaaacgtcagctggttgagacccgtcaaatcaccaagcatgttgcgcagatcctggacagccgtatgaatactaagtacgacgagaacgataagcttatccgtgaggtgaaggtgatcaccttgaaatcgaaactggtgagcgactttcgcaaagacttccagttttataaggtacgtgaaatcaacaactatcaccacgcacatgatgcatatctgaatgctgtggttggcaccgcgttgattaaaaagtacccgaaattggagagcgagttcgtgtatggtgattacaaggtctatgacgttcgtaagatgattgcgaaaagcgagcaggagattggcaaagcaaccgctaaatactttttctattccaacattatgaatttcttcaagactgaaatcaccctggcgaatggtgagattcggaaacgtccgctgatcgagacgaacggtgaaaccggtgaaatcgtgtgggataagggccgtgatttcgcgaccgtgcgtaaggtgctaagcatgccgcaggttaatatcgtgaagaaaacggaggttcagaccggtggtttcagcaaggagagcatcctcccgaaacgcaattccgataagctgattgcacgtaagaaggactgggatccgaaaaagtacggcggctttgactcgccgaccgtggcatacagtgttttggtagttgccaaggtggaaaagggcaaaagcaaaaagcttaagagcgtgaaggagttgctgggtataaccataatggaacgaagtagcttcgagaagaacccgatcgatttcttggaggcgaaaggctacaaagaagtgaagaaggacctgattatcaagttaccgaaatacagcctgtttgaacttgaaaacggccgtaagcggatgctggcgagcgcgggcgaacttcagaaagggaacgagctcgcgctaccgtccaaatacgtgaactttctttatctggccagccattacgaaaagctgaagggttccccggaagataacgagcaaaaacagctcttcgtcgagcagcataaacactacttggacgagattatagaacagattagcgagttcagcaagcgtgtgatcttggcggatgccaacctggacaaggttctgagcgcgtataacaaacatcgtgataagccgattcgtgaacaggcagagaacattatccacctgttcaccttaaccaatctgggagctccggcagcatttaaatacttcgacaccaccattgatcgtaagcgttatacctcgacgaaagaggttttggacgcgactctgatccaccaatccattaccggcctgtacgaaactcgcattgatttatcccaattaggtggtgacaagaggcccgcggcaacaaaaaaagctggacaggcgaaaaagaagaaggactacaaagatgacgacgataaataactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaagacagaagagcaatctctagaggatcccc

SEQ ID NO: 4 - DNA, 8476 bp, synthetic construct
  source 1..8476: mol_type = other DNA
taatacgactcactatagatgggcggcgcatgagagaagcccagaccaattacctacccaaaatggaaaaagttcacgtagatatagaggaagacagcccgttcctgcgtgctctccagcgttcgttcccacagtttgaagtggaagcgaagcaagttaccgataatgatcatgcgaatgcgagagcgttctcccatctggcgagcaaactgattgaaactgaggtggacccgagcgataccatcctggacatcggcagcgcaccagctcgccgtatgtatagcaagcacaaataccactgtatttgtccgatgcgttgcgccgaggacccggaccgcctgtacaaatatgcgacgaaattgaagaagaactgcaaagagattaccgacaaagaactggataaaaagatgaaagagctggcggcggttatgagcgacccggacttagagacggaaacgatgtgcctgcatgatgacgagagctgtcgttatgaaggtcaggtcgcagtttatcaggatgtctacgcggttgacggtccgacctccctgtaccaccaggcgaacaaaggcgtgcgcgtggcctactggatcggttttgatacaaccccgttcatgtttaagaacttggcaggcgcttacccgtcttactccacgaactgggccgatgagaccgttttgaccgcgcgcaacatcggcctctgcagcagcgacgtaatggaacgttctagacgcggcatgtcgatcctgcgcaagaagtacctgaagccgtccaataatgttctgttcagcgttggtagtaccatttaccacgagaaacgtgatctgctgcgtagctggcatctgccgtccgtgttccacttgagaggtaaacaaaactacacctgtcgctgcgaaaccatcgttagctgcgatggctacgttgtgaaacgcatcgcaattagcccgggtctgtatggtaagccttctggttatgccgcgacgatgcaccgtgaaggttttctgtgctgcaaggtgaccgataccctgaacggtgaacgcgtctcgttcccggtgtgtacctatgtgccggctacgttgtgtgatcaaatgaccggtattctggcaaccgacgtgagcgcggacgacgcccagaaacttctggtgggcttgaaccagcgtattgttgtgaacggccgtacccaacgtaacactaatacgatgaagaactatctgttgccagtagtggcccaagcattcgcgcgttgggcgaaggagtataaagaggaccaagaagatgaacgtccgctgggtcttcgcgaccgtcagctggttatgggttgctgctgggcatttcgtcgccataaaattaccagcatctacaagcgtccggatacccagaccatcatcaaagttaattctgattttcacagctttgttctgccgcgtatcggcagcaacactctagagatcggcttgcggacccgcattcgtaagatgctggaagagcacaaggagccgtcaccgttaatcactgcggaagatgtccaggaggcaaagtgcgcggctgacgaagctaaggaggttcgtgaagctgaggagttgcgtgcggcgctgcctccgttggcggcggatgtggaagagccgaccttggaggctgacgtggacctaatgctgcaagaggctggtgccggatcagtagaaacacccagggggctaataaaggtcacaagctatgacggcgaagataaaatcggtagttacgctgttttatctcctcaagcggttctgaagtccgagaagcttagctgtattcatccgttagccgagcaggttattgtcatcacccacagcggtcgcaaaggccgctacgcggttgagccgtatcacggcaaggtggtggttccggagggtcatgctatcccggttcaggatttccaagcgttaagcgaaagcgcgaccatcgtgtacaatgaacgtgaattcgtaaaccgctatctgcaccacattgcgacgcacggtggcgcgctgaacaccgatgaagagtactacaaaaccgtcaaaccgtcagagcacgacggtgagtatctgtacgacatcgaccgtaaacagtgtgttaagaaggagctggttaccggtctgggtctgacgggcgagctggtagatccgccttttcatgagttcgcatatgaaagcctgcgcactcgcccggcagcgccgtatcaagtgccgacgatcggcgtctacggcgttccgggtagcggtaagtccggtattattaagtctgccgttaccaagaaagatttagtcgtttccgcgaaaaaagagaactgcgcagaaatcattcgtgatgttaaaaagatgaagggactggacgtaaacgctcgtacggtggacagcgtgttgctgaatggttgcaaacatccggtggagaccctgtacatcgacgaggcgtttgcatgtcatgccgggactctgcgcgcgttgatagctatcatccgtccgaagaaggcggtgttgtgcggtgatccgaaacagtgcggttttttcaacatgatgtgcctgaaagttcattttaaccacgagatctgcacccaggtgtttcacaaatcgattagccgtcgttgtaccaaatccgttaccagcgttgtgagcaccttgttctacgataaaaaaatgcgtacgaccaacccgaaagaaaccaagatcgttattgataccacgggttcaaccaagccgaagcaagatgatttgatcctgacctgctttcgcggttgggttaagcagctgcagattgactacaagggcaacgaaattatgaccgcagctgcttcacaaggcctgacaagaaaaggcgtttatgcggtgcgttacaaggttaacgagaatccgctgtatgccccaaccagcgagcacgtgaacgttttgctgacccgtaccgaggaccgcatcgtttggaaaacgctggcaggcgacccgtggattaagaccctgaccgcgaaatacccgggcaacttcaccgcgaccattgaagagtggcaggctgagcacgatgcaattatgcgccatattctggaacgtccggatccgacggatgtgttccagaacaaagccaatgtttgttgggctaaggcgctcgtgccagtgcttaagaccgcgggcatcgatatgactactgaacaatggaataccgtcgactacttcgaaaccgacaaggcacattctgcggagatcgttctgaatcagctgtgcgtacgtttttttggtttggacctggactcgggtctgttctccgctccgaccgtacccctgtctatccgcaacaatcattgggataatagcccgagtccaaatatgtatggtctgaacaaagaagttgttcgccaactgtctcgtcgctacccgcagttaccgcgtgccgtggcgactggcagggtatacgacatgaataccggcaccttgcgtaactatgaccctcgtattaacctggtcccggtgaatcgtcgcctgccgcatgccttggttcttcaccacaatgaacacccgcaaagcgacttttccagcttcgtcagcaaactgaaaggtcggacggttttagtcgtgggtgaaaaactgtctgttccgggcaaaatggttgactggctgtccgatcgtccggaagcgacctttcgtgcgcgtctagacctgggtattccgggtgacgtgccaaaatacgacatcatctttgtcaacgtgcgcacaccatataagtaccatcattatcagcagtgtgaagaccacgcgattaagctgagcatgctgaccaagaaggcctgcctgcacctgaacccgggtggtacgtgcgtgtcgatcggttacggctacgcagatagagctagcgagtcgatcatcggtgcaatcgctcgtcaattcaaattcagccgtgtgtgcaagccgaaatccagcttggaagaaaccgaggtgttgttcgtgttcatcggctatgaccgtaaggcccgtacgcacaacagctacaagttgtctagcactctgaccaacatttataccggcagccgtctccacgaagctggctgcgctccctcatatcacgtagttaggggagatatcgctaccgcaaccgagggtgtgattattaatgcggcgaactccaaaggtcagccgggtggtggcgtttgcggcgcgctgtacaaaaaatttccggaatcgttcgacctgcaaccgatcgaagtgggtaaagcacgcctggtaaaaggcgcggcaaaacacattatccacgcagtgggcccaaattttaacaaggtgagcgaagtcgaaggcgacaagcaactggctgaagcgtatgagtccatagcaaagatcgtgaacgataacaactataagagcgttgccatcccgttgctttctacgggtattttttccggcaataaagatcgtttaacccagagcctgaatcacctgcttaccgcattagacacgaccgatgccgatgttgcgatttactgccgtgataaaaagtgggaaatgaccctgaaggaagctgtggctcgtcgtgaagcggttgaagagatttgcatcagcgatgatagctccgtgacagaaccggacgcggagttggttcgcgttcatccgaagagctcgctggcgggtcgtaaaggttatagcaccagcgatggtaagacctttagctatttggaaggtaccaaattccaccaggcggcgaaggacattgctgagatcaatgctatgtggcctgtcgctaccgaggcgaacgaacaagtgtgcatgtatattctgggcgagtcaatgagctcaattcgtagcaagtgcccggttgaagagtctgaagccagcaccccgccgagcaccttaccgtgtctgtgtatccacgccatgacccccgaacgtgttcagcgtttgaaggcaagtcgtccggaacagatcaccgtttgcagctcattcccgttgccgaaataccggattaccggcgtgcagaaaatccaatgtagccagccgatcttgttctccccgaaagttccggcgtacatccacccgcgtaagtacttggttgagaccccgccggtggacgaaacgccggaacccagcgcagaaaaccagtctactgaaggcaccccagagcaaccgccactgatcaccgaggacgagacccgcacgcgtactccagagccgatcatcatcgaggaggaagaggaggacagcattagcctgctgagcgacggtccgacccatcaagttctgcaggttgaggccgacattcacggtccgccaagcgtgtcttcgagcagttggagcattccgcatgcatctgacttcgacgtggattccctgtccatcctggataccctggagggcgcctccgtcaccagtggtgcgacctctgcggaaacgaacagctacttcgcgaagagtatggaatttctggcgcgtccggtaccggcccctcgcactgtatttcgtaacccgccgcatccggctccgcgtacgagaactccgtccctcgctccgtcgcgcgcgtgcagccgcacgagcttggtttccaccccgccgggagttaaccgcgtgatcacccgtgaggagctggaggccctgaccccgtcgcgcacgccgagccgttccgtttcaagaacctccctggtgtctaatccgccgggcgtcaaccgagtcattacccgtgaggaattcgaggcattcgtggcgcagcaacaaagccgttttgatgctggcgcgtatatattttcaagtgatacaggacaagggcacctgcagcagaaatctgttcgtcagaccgtgctgagcgaggttgttttggagcgcacggaattggagatcagctacgcaccgcgtcttgaccaagaaaaagaggaattgctgcgcaagaagctgcaactgaacccgacgccggcaaataggtcccgttatcagagccgtaaagttgagaacatgaaagcgatcaccgcgcgtcggattctgcaaggtctgggccattatctgaaggcagagggtaaagtggagtgctatcgtaccctgcatccggttccgctgtacagctcctctgttaaccgcgccttcagctctccgaaggtcgccgtcgaggcgtgcaatgctatgctgaaagaaaactttccaacggtagcgagctactgcattattccggaatacgacgcgtacttggacatggtggatggtgccagctgttgtctggataccgcatccttctgcccggcaaaactgcgtagcttcccgaaaaaacacagctatttagagccgaccattcgtagcgctgtgccgagcgccatccagaacaccttgcaaaacgttctcgcggccgctaccaagcgcaattgtaatgtgacccagatgcgtgagctgccagttctggattctgcagcgttcaacgtggagtgctttaaaaagtatgcctgcaacaacgaatactgggaaacctttaaagagaaccctattcgcctgaccgaggagaacgtcgtgaattatatcaccaagttgaagggtccgaaggcggcggccctgttcgccaaaacccataatctgaacatgctgcaagacatcccgatggatcgttttgttatggacctgaaaagagatgtgaaggtcactccgggtacgaagcacaccgaagagcgccctaaggtccaggttattcaagcagctgatccgctggcaaccgcgtacttatgcggtatccaccgtgaattggttcgtcgtttaaacgcagttttgttgcccaacattcacaccctgtttgatatgagcgcggaggatttcgacgcgattatcgccgagcactttcagccgggtgactgtgttctggagacggatattgcctcgttcgacaaatccgaggacgacgcgatggcgttgactgcgctgatgattttggaggatctgggcgttgatgcggaactgctgactctgatcgaagcggcgttcggtgagattagctcgatccacttgccgaccaagaccaagttcaaattcggtgcgatgatgaagtccggcatgtttctcacccttttcgtgaacaccgttatcaatatcgtgatcgcctctcgtgttctgcgcgaacgcctcacgggctcgccgtgtgcggcttttattggcgacgacaacatcgtgaagggcgttaaaagtgataaactgatggcggaccgttgtgcaacctggctgaatatggaagtgaaaattatcgatgcggtcgtaggcgaaaaggctccgtatttctgcggtggttttatcctgtgcgatagcgtgactggtacagcgtgccgtgtggctgacccgctgaagcgcttattcaagttgggcaaaccgctggcggcggatgacgaacatgacgacgacagacgtcgtgcactgcatgaagagtcgacccgttggaatcgtgtcggcatcctaagcgagctctgcaaagcggttgaatcacgctacgaaacggtggggacctccatcatcgtgatggctatgaccaccctggcgtctagtgtgaagagctttagctacctgcgtgggcccctataactctctacggctaacctgaatggactacgacatagtctagtccgccaagatggttagtagggtatcaaaaggagaagagttgtttaccggtgttgttccgatcctggtggaactggatggcgacgttaacggtcacaagttctccgtgagcggtgagggcgagggcgacgctacctacggtaaattgaccttgaagttcatctgtaccacgggtaaactgccagttccgtggccgaccttagtaacaactctgacgtacggcgtgcaatgcttttctcgttatccggaccacatgaaacagcatgatttcttcaagtccgccatgccggaaggttatgttcaagagcgcaccatttttttcaaggacgacggcaactacaagacccgtgcggaagttaaattcgagggtgataccctcgtcaatcgtattgagcttaagggtatcgattttaaagaagacggcaatattctgggtcacaaattggagtataactacaacagccataatgtttatatcatggcagataaacaaaaaaacggcatcaaggtgaactttaagatccgccataacatcgaggacggcagcgtccagctggcggaccactaccagcagaacaccccgattggtgacggaccggtgctgctgccggataatcattacctgtcgacccagagcgcgctgagcaaggatccgaatgaaaaacgtgatcacatggtgttgctggaattcgtgacggctgcgggtattaccctgggcatggatgaactgtataagtaatagatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaagctcttcc

(19) United States
(12) Patent Application Publication
(10) Pub. No.: US 2025/0215405 A1
(43) Pub. Date: Jul. 3, 2025

(54) T7 RNA POLYMERASE MUTANTS AND THEIR APPLICATIONS

(71) Applicant: Yeasen Biotechnology (Shanghai) Co., Ltd., Shanghai (CN)

(72) Inventors: Xiang Liu, Pudong New District (CN); Qiongwei Tang, Pudong New District (CN); Sisi Zhu, Pudong New District (CN); Nannan Hu, Pudong New District (CN); Sainan Yin, Pudong New District (CN); Wei Xie, Pudong New District (CN); Dongliang Song, Pudong New District (CN)

(21) Appl. No.: 18/772,730

(22) Filed: Jul. 15, 2024

Related U.S. Application Data
(60) Provisional application No. 63/655,458, filed on Jun. 3, 2024.

(30) Foreign Application Priority Data
Dec. 28, 2023 (CN) 2023118375063
Apr. 8, 2024 (CN) 2024104181762

(51) Int. Cl. C12N 9/12; C12P 19/34

(52) U.S. Cl. CPC C12N 9/1247; C12P 19/34; C12Y 207/07006

(57) ABSTRACT

The present disclosure provides mutant T7 RNA polymerase proteins obtained through the introduction of one or more amino acid mutations (e.g., substitutions) at different sites based on the wild-type T7 RNA polymerase. Compared to the wild type T7 RNA polymerase, these mutants exhibited one or more of the following characteristics: lower levels of dsRNA by-products, higher product integrity, increased yield, or increased transcriptional activity. The mutant T7 RNA polymerase proteins of the present disclosure, with reduced levels of dsRNA by-products, are more suitable for in vitro transcription systems than compared to the wild-type T7 RNA polymerase. The significantly low levels of by-products and higher levels of purity allow for the requirements of RNA-based agents or mRNA vaccines to be readily met through simple purification, significantly reducing downstream purification complexity and costs. Additionally, the transcription products are of high quality and may have lower immune side effects.

### CROSS REFERENCE TO RELATED APPLICATIONS

This application claims priority and benefit from U.S. Provisional Patent Application No. 63/655,458, filed Jun. 3, 2024, Chinese Patent Application No. 2024104181762, filed Apr. 8, 2024, and Chinese Patent Application No. 2023118375063, filed Dec. 28, 2023, the contents of each of which are hereby incorporated by reference in their entireties.

### SEQUENCE LISTING

The instant application contains a Sequence Listing which has been submitted electronically in XML format and is hereby incorporated by reference in its entirety. Said XML copy, created on Jul. 15, 2024, is named 000477-00001-101-SL.xml and is 21,244 bytes in size.

### TECHNICAL FIELD

The disclosures involve T7 RNA polymerase mutants and their applications in transcribing deoxyribonucleic acids (DNA).

### BACKGROUND

Ribonucleic acid (RNA) is an important biological macromolecule that plays a crucial role in the transmission of genetic information. In recent years, with deepening research into RNA, the role of RNA in disease detection and treatment has gained prominence, leading to the rise of RNA therapies. RNA therapy refers to the use of RNA-based molecules for the treatment or prevention of diseases. RNA therapy has high target specificity, low genotoxicity, and holds great potential for treating or preventing a wide range of genetic diseases and some rare conditions. With the successful development of mRNA COVID-19 vaccines and the approval of various other novel RNA-based agents, RNA-based agents are increasing in the forefront of drug research. This has posed a significant challenge in supplying high-quality RNA molecules of specific lengths and sequences. There are two methods for in vitro synthesis of RNA, i.e., chemical synthesis and enzymatic synthesis. Chemical synthesis is primarily suitable for short-chain RNA, whereas the high cost of synthesis limits its practicality for long-chain RNA. Protein-encoding mRNAs typically consist of several thousand nucleotides. Therefore, for long-chain mRNA, enzymatic synthesis is currently the common and optimal approach.

In vitro synthesis of long-chain mRNA typically involves using a double-stranded DNA template, which is transcribed into mRNA by an RNA polymerase. For this transcription, T7 RNA polymerase is the most commonly used RNA polymerase. T7 RNA polymerase, derived from the T7 bacteriophage of Escherichia coli, is a single-subunit RNA polymerase that was identified in the 1970s. Since then, it has been widely used in in vivo protein expression and in vitro RNA synthesis. T7 RNA polymerase consists of 883 amino acids in total, weighs approximately 99 kDa, uses double-stranded DNA as a template, and transcribes and synthesizes mRNA by recognizing specific promoter sequences. T7 RNA polymerase has a mature and stable heterologous expression and purification process. It can function with full transcriptional activity without requiring any additional cofactors, and it can produce high-fidelity, full-length RNA transcripts. Therefore, it has become the most mainstream tool enzyme for in vitro transcription. Although T7 RNA polymerase has significant advantages as an in vitro RNA synthesis enzyme, it still has some shortcomings in such synthetic applications. For example, during the in vitro synthesis of RNA, in addition to the target product, such as the full-length single-stranded RNA, T7 RNA polymerase also produces several other by-products, including dsRNA, short single-stranded RNA, and 3′-end extension products. However, mRNA vaccines or other RNA-based therapeutic agent have stringent requirements for RNA purity and integrity, so as to minimize immune responses in the body and to achieve optimal efficacy and safety. Although transcription by-products can be removed through purification and other means to improve RNA quality, these steps also increase costs and operational complexity, which is not conducive to large-scale production. Therefore, there is an unmet need to reduce by-products during the transcription reaction using T7 RNA polymerase and to obtain high-quality RNA products in a more direct, economical, and effective way.

To address these issues, the present disclosure provides mutant T7 RNA polymerase proteins capable of obtaining higher quality RNA in in vitro synthesis, as well as compositions comprising those mutant proteins and methods of using them in the transcription of DNA.

### SUMMARY OF THE DISCLOSURE

The objective of the present disclosure is to provide mutant T7 RNA polymerase proteins and compositions comprising them and methods of using them to transcribe DNA.

In some embodiments, this disclosure provides:
A mutant T7 RNA polymerase protein, wherein the mutant protein is selected from one of the proteins described in a1-a4 below:
a1: a protein having an amino acid substitution at one or at least two of the following positions of the wild-type T7 RNA polymerase whose amino acid sequence is as shown in SEQ ID NO.1: 16, F11, 119, K60, N67, A70, 182, N86, D87, F162, K180, V186, V214, N233, A247, P277, M369, Y457, A465, H523, K610, A615, L651, S686, K740, or N764;
a2: a protein with substantially similar enzymatic activity and function, obtained by substituting and/or deleting and/or adding one or more amino acid residues to the amino acid sequence shown in a1, in addition to the mutations described above;
a3: a protein with at least 90% sequence homology to protein a1, and with enzymatic activity and function substantially equivalent to protein a1; or
a4: a fusion protein obtained by connecting the N-terminus and/or C-terminus of any one of the amino acid sequences described in a1-a3 with a tag or enzyme cleavage site, where the tag or enzyme cleavage site does not affect the function of the mutant T7 RNA polymerase protein.

In some embodiments, the mutant T7 RNA polymerase protein is any one of the proteins described in a1-a4 below:
a1: a protein that is substituted at one or at least two of the following positions of the wild-type T7 RNA polymerase whose amino acid sequence is as shown in SEQ ID NO.1: 16, 119, K60, N67, A70, N86, F162, K180, V186, N233, A247, P277, A465, L651, or N764;
a2: a protein with substantially similar enzymatic activity and function, obtained by substituting and/or deleting and/or adding one or more amino acid residues to the amino acid sequence shown in a1, in addition to the mutations described above;
a3: a protein with at least 90% sequence homology to protein a1, and with enzymatic activity and function substantially equivalent to protein a1; or
a4: a fusion protein obtained by connecting the N-terminus and/or C-terminus of any one of the amino acid sequences described in a1-a3 with a tag or enzyme cleavage site, where the tag or enzyme cleavage site does not affect the function of the mutant T7 RNA polymerase protein.

In some embodiments, the respective amino acid residue is replaced with a combination of any one or at least two of the following: I6G, F11L, I19T, K60I, N67S, A70Q, or T, I82V, N86D, D87G, F162S, K180E or D, V186I, V214A, N233D, A247T or I, P277L, M369S or T, Y457H, A465T, H523R, K610R, A615T, L651M, S686G, K740R, or N764D.

In some embodiments, the respective amino acid residue is replaced with a combination of any one or at least two of the following: 16G, F11L, I19T, K60I, N67S, A70Q, N86D, F162S, K180E or D, V186I, V214A, N233D, A247I, P277L, M369S, Y457H, A465T, H523R, L651M, or N764D.

In some embodiments, the mutant T7 polymerase protein is selected from any one of the mutant proteins described in b1-b3 below:
b1: a protein obtained by substituting any combination of amino acids on the basis of the wild-type T7 RNA polymerase amino acid sequence as shown in SEQ ID NO.1:
(1) N86D/A615T;
(2) F162S;
(3) F162S/K180E;
(4) A247I;
(5) P277L;
(6) A70Q/F162S/K180E;
(7) N233D/A465T;
(8) D87G/N233D/V186I;
(9) A70T/P277L;
(10) N67S/I82V/Y457H/K610R/L651M/N764D;
(11) F162S/A247T;
(12) F162S/N233D/A247T;
(13) V214A;
(14) I19T/K180D/M369T;
(15) F162S/K180E/A247I;
(16) A70Q/V214A;
(17) A465T;
(18) F11L;
(19) K180E;
(20) M369S/S686G;
(21) K60I/A465T/K740R;
(22) H523R;
(23) A70T/K180D;
(24) I6G;
(25) A70Q/K180E;
(26) A70Q;
(27) V214A/A465T; or
(28) A70Q/A247T;
b2: a protein with substantially similar enzymatic activity and performance, obtained by substituting and/or deleting and/or adding one or more amino acid residues other than the mutations mentioned above to the amino acid sequence shown in b1; or
b3: a fusion protein obtained by connecting the N-terminus and/or C-terminus of any one of the amino acid sequences described in b1-b2 with a tag or enzyme cleavage site, where the tag or enzyme cleavage site does not affect the function of the T7 RNA polymerase mutant.

The aforementioned mutant T7 RNA polymerase proteins exhibit one or more of the following characteristics when used in the transcription of DNA: lower amounts of dsRNA by-products (typically ≤0.01 ng/g; or ≤0.001 ng/g; or ≤0.0001 ng/g), higher product integrity (an improvement of ≥5%, or ≥9%), increased yield or transcriptional activity, or reduced usage of cap analogs.

In some embodiments, a His tag and thrombin cleavage site are linked at the N-terminus of the mutants.

The coding gene of the aforementioned T7 RNA polymerase mutants.

The expression vector of the aforementioned T7 RNA polymerase mutants.

The expression host bacteria of the aforementioned T7 RNA polymerase mutants.

The application of the aforementioned T7 RNA polymerase mutants in the preparation of in vivo transcription or in vitro transcription reagents.

The present disclosure further discloses a transcription reagent kit containing the aforementioned T7 RNA polymerase mutants.

The application of the aforementioned T7 RNA polymerase mutants in catalyzing co-transcriptional capping reactions.

The present disclosure further discloses a co-transcriptional capping reagent kit comprising the aforementioned T7 RNA polymerase mutants, a cap analog, and a buffer system.

In some embodiments, the buffer system comprises: 30-50 mM Tris, 5-20 mM DTT, 1-5 mM spermidine, and 20-100 mM MgCl₂.

The present disclosure also discloses a method comprising: contacting a nucleic acid (e.g., DNA) template with the aforementioned T7 RNA polymerase mutants to transcribe and obtain transcription products.

In some embodiments, the method of this disclosure further comprises contacting the transcription products with pharmaceutically acceptable additives to produce a pharmaceutical dosage form.

In some embodiments, the method of this disclosure further comprises contacting the transcription products with capping enzymes to form capped transcription products.

In some embodiments, the method of this disclosure further comprises contacting the capped transcription products with pharmaceutically acceptable additives to produce a pharmaceutical dosage form.

The present disclosure also discloses a method comprising: contacting a nucleic acid template, a cap analog, the aforementioned T7 RNA polymerase mutants, as well as a buffer system, to perform transcription and capping to obtain capped transcription products.

In some embodiments, the method of this disclosure further comprises contacting the capped transcription products with pharmaceutically acceptable additives to produce a pharmaceutical dosage form.

The present disclosure provides mutant T7 RNA polymerase proteins obtained through the introduction of one or more amino acid mutations at different sites based on the wild-type T7 RNA polymerase. Compared to the wild type, these mutants exhibit one or more of the following characteristics when used in the transcription of nucleic acids: lower amounts of dsRNA by-products, higher product integrity, and increased yield or transcriptional activity. The T7 RNA polymerase mutants of the present disclosure, with reduced amounts of dsRNA by-products, are more suitable for in vitro transcription systems than the wild-type T7 RNA polymerase. Their extremely low amounts of by-products and higher levels of purity allow for the requirements of RNA-based agents or mRNA vaccines to be readily met through simple purification, significantly reducing downstream purification complexity and costs. Additionally, the transcription products of this disclosure are of high quality and have lower immune side effects than transcription products generated using wild-type T7 RNA polymerases. The T7 RNA polymerase mutants of the present disclosure may also include one or more of the following features: higher product integrity, increased transcription yield, enhanced transcriptional activity, reduced usage of cap analogs, etc.

Compared to the prior art, the present disclosure has the following benefits:
(1) The mutant T7 RNA polymerase proteins of the present disclosure are capable of significantly reducing the content of dsRNA by-products during in vitro transcription.
(2) The T7 RNA polymerase mutants of the present disclosure have higher product integrity.
(3) The T7 RNA polymerase mutants of the present disclosure have higher transcriptional activity, with the optimal effect up to twice that of the wild type.
(4) The T7 RNA polymerase mutants of the present disclosure are suitable for low concentrations of cap analogs, allowing for a significant reduction in the amount of cap analogs used and thereby saving costs for in vitro transcription.

Some embodiments of this disclosure are:
1. A mutant T7 RNA polymerase protein selected from the group of:
a1: a protein comprising a substitution at one or at least two of the amino acid residues of a wild-type T7 RNA polymerase comprising the amino acid sequence set forth in SEQ ID NO:1, the amino acid residues being selected from the group of:

### I6, I19, K60, N67, A70, N86, F162, V186, N233, A247, P277, A465, L651, or N764; or

a2: a protein comprising an amino acid sequence having at least 90% sequence identity to a mutant T7 RNA polymerase protein of a1, and whose activity and enzymatic properties are substantially the same as one or more of a mutant T7 RNA polymerase protein of a1.
2. The mutant T7 RNA polymerase protein according to embodiment 1, wherein the protein further comprises a substitution at one or at least two of the amino acid residues selected from the group of F11, I82, D87, K180, V214, M369, Y457, H523, K610, A615, S686, or K740.
3. The mutant T7 RNA polymerase protein according to embodiment 1 or 2, wherein the substituted protein further comprises one or more of an additional substitution, deletion, or addition of a mutant T7 RNA polymerase protein of embodiment 1 or embodiment 2, the protein having substantially the same activity and enzymatic properties as one or more of a mutant T7 RNA polymerase protein of embodiment 1 or embodiment 2.
4. The mutant T7 RNA polymerase protein according to any one of embodiments 1 to 3, wherein the protein comprises one or a combination of at least two of an amino acid substitution selected from the group of: I6G, F11L, I19T, K60I, N67S, A70Q, A70T, I82V, N86D, D87G, F162S, K180E, K180D, V186I, V214A, N233D, A247T, A247I, P277L, M369S, M369T, Y457H, A465T, H523R, K610R, A615T, L651M, S686G, K740R, or N764D.
5. The mutant T7 RNA polymerase protein according to any one of embodiments 1 to 3, wherein the protein comprises one or a combination of at least two of an amino acid substitution selected from the group of: I6G, F11L, I19T, K60I, N67S, A70Q, N86D, F162S, K180E, K180D, V186I, V214A, N233D, A247I, P277L, M369S, Y457H, A465T, H523R, L651M, or N764D.
6. A mutant T7 RNA polymerase protein selected from the group of:
b1: a protein comprising a substitution at one or at least two of the amino acid residues of a wild-type T7 RNA polymerase comprising the amino acid sequence set forth in SEQ ID NO:1, the amino acid residues being selected from the group of:

### I6G, F11L, I19T, K60I, N67S, A70Q, A70T, N86D, F162S, K180E, K180D, V186I, V214A, N233D, A247T, A247I, P277L, M369S, Y457H, A465T, H523R, L651M, or N764D;

b2: a protein comprising a substitution at least two of the amino acid residues of a wild-type T7 RNA polymerase comprising the amino acid sequence set forth in SEQ ID NO:1, the amino acid residues being selected from the group of:

### I82V, D87G, M369T, K610R, A615T, S686G, or K740R;

b3: the protein of b1 further comprising a substitution at one or at least two of the amino acid residues of a wild-type T7 RNA polymerase comprising the amino acid sequence set forth in SEQ ID NO:1, the amino acid residues being selected from the group of:

### I82V, D87G, M369T, K610R, A615T, S686G, or K740R; or

b4: a protein comprising an amino acid sequence having at least 90% sequence identity to a mutant T7 RNA polymerase protein of b1, b2, or b3, and whose enzymatic activity and properties are substantially the same as one or more of a mutant T7 RNA polymerase protein of b1, b2, or b3.
7. A mutant T7 RNA polymerase protein selected from the group of:
c1: a protein comprising a substitution at one or at least two of the amino acid residues of a wild-type T7 RNA polymerase comprising the amino acid sequence set forth in SEQ ID NO:1, wherein the substitution is selected from the group of:
(1) N86D/A615T;
(2) F162S;
(3) F162S/K180E;
(4) A247I;
(5) P277L;
(6) A70Q/F162S/K180E;
(7) N233D/A465T;
(8) D87G/N233D/V186I;
(9) A70T/P277L;
(10) N67S/I82V/Y457H/K610R/L651M/N764D;
(11) F162S/A247T;
(12) F162S/N233D/A247T;
(13) V214A;
(14) I19T/K180D/M369T;
(15) F162S/K180E/A247I;
(16) A70Q/V214A;
(17) A465T;
(18) F11L;
(19) K180E;
(20) M369S/S686G;
(21) K60I/A465T/K740R;
(22) H523R;
(23) A70T/K180D;
(24) I6G;
(25) A70Q/K180E;
(26) A70Q;
(27) V214A/A465T; or
(28) A70Q/A247T; or
c2: a protein comprising an amino acid sequence having at least 90% sequence identity to a mutant T7 RNA polymerase protein of c1, and whose activity and enzymatic properties are substantially the same as one or more of a mutant T7 RNA polymerase protein of c1.
8. The mutant T7 RNA polymerase protein according to embodiment 7, wherein the substituted protein further comprises one or more of an additional substitution, deletion, or addition of a mutant T7 RNA polymerase protein of embodiment 7, the protein having substantially the same activity and enzymatic properties as one or more of a mutant T7 RNA polymerase protein of embodiment 7.
9. The mutant T7 RNA polymerase protein according to any one of embodiments 1 to 8, wherein the protein further comprises one or more of a tag or an enzyme cleavage site at one or both of the N-terminus or the C-terminus of the protein, and wherein the tag or the enzyme cleavage site does not substantially affect the activity and enzymatic properties of the mutant T7 RNA polymerase protein.
10. The mutant T7 RNA polymerase protein according to embodiment 9, wherein the tag is a His tag and the enzyme cleavage site is a thrombin cleavage site.
11. The mutant T7 RNA polymerase protein according to embodiment 10, wherein one or more of the His tag or the thrombin cleavage site is at the N-terminus of the protein.
12. The mutant T7 RNA polymerase protein according to any one of embodiments 1 to 11, wherein the protein, as compared to a wild-type T7 RNA polymerase comprising the amino acid sequence set forth in SEQ ID NO:1, is characterized by producing during transcription one or more of a lower amount of dsRNA by-products, a higher product integrity, a higher yield, a higher transcriptional activity, or a lower cap analog usage.
13. The mutant T7 RNA polymerase protein according to any one of embodiments 1 to 12, wherein the protein is characterized by being capable of catalyzing co transcriptional capping.
14. A composition comprising the mutant T7 RNA polymerase protein according to any one of embodiments 1 to 13, and one or more of an in vivo transcription reagent or in vitro transcription reagent.
15. A transcription kit comprising a mutant T7 RNA polymerase protein according to any one of embodiments 1-13 or a composition according to embodiment 14.
16. The transcription kit according to embodiment 15, wherein the transcription kit is a co-transcription capping kit.
17. The transcription kit according to embodiment 16, further comprising a cap analog and a buffer system.
18. The transcription kit according to embodiment 17, wherein the buffer system comprises: 30-50 mM Tris, 5-20 mM DTT, 1-5 mM spermidine, and 20-100 mM MgCl2.
19. A method of generating a transcription product of a deoxyribonucleic acid (DNA) comprising contacting the DNA with a mutant T7 RNA polymerase protein according to any one of embodiments 1 to 13 or a composition according to embodiment 14.
20. The method according to embodiment 19, further comprising contacting the transcription product with a capping enzyme to form a capped transcription product.
21. The method according to embodiment 19 or 20, further comprising combining the transcription product or the capped transcription product with a pharmaceutically acceptable additive to produce a pharmaceutical dosage form.
22. A method of generating a capped transcription product of a deoxyribonucleic acid (DNA) comprising:
(i) mixing the DNA, a cap analog, and a mutant T7 RNA polymerase protein according to any one of embodiments 1 to 13, or a composition according to embodiment 14, in a buffer system; and
(ii) generating the capped transcription product through transcription of the DNA using the mixture of step (i).
23. The method of embodiment 22, further comprising combining the capped transcription product with a pharmaceutically acceptable additive to produce a pharmaceutical dosage form.

### BRIEF DESCRIPTION OF DRAWINGS

FIG. 1: Electropherograms of purified proteins of T7 RNA polymerase mutants Mut-022, Mut-025, Mut-029, Mut-049, Mut-050, and Mut-057. In the figure, M022 refers to Mut-022, which similarly applies to the other mutants in this figure and subsequent figures; 1 #, 2 #, and 3 #represent different sample loading volumes, the same below.

FIG. 2: Electropherograms of purified proteins of T7 RNA polymerase mutants Mut-077, Mut-082, Mut-083, Mut-103, and Mut-119.

FIG. 3: Electropherograms of purified proteins of T7 RNA polymerase mutants Mut-134, Mut-139, Mut-144, Mut-161, Mut-162, and Mut-168.

FIG. 4: Electropherograms of purified proteins of T7 RNA polymerase mutants Mut-176, Mut-180, Mut-184, Mut-195, Mut-197, Mut-207, Mut-218, and Mut-231.

FIG. 5: Electropherograms of purified proteins of T7 RNA polymerase mutants Mut-246, Mut-258, and Mut-271.

FIG. 6: Dot blot hybridization diagrams of dsRNA products of T7 RNA polymerase mutants Mut-022, Mut-025, Mut-029, Mut-049, Mut-050, Mut-057, Mut-077, and Mut-082. WT represents wild-type T7 RNA polymerase, with each mutant undergoing four parallel experiments, the same below.

FIG. 7: Dot blot hybridization diagrams of dsRNA products of T7 RNA polymerase mutants Mut-083, Mut-103, Mut-119, and Mut-134. WT represents wild-type T7 RNApolymerase.

FIG. 8: Dot blot hybridization diagrams of dsRNA products of T7 RNA polymerase mutants Mut-139, Mut-144, Mut-161, Mut-162, and Mut-168. WT represents wild-type T7 RNA polymerase.

FIG. 9: Dot blot hybridization diagrams of dsRNA products of T7 RNA polymerase mutants Mut-176, Mut-180, Mut-184, Mut-195, Mut-197, Mut-207, and Mut-218. WT represents wild-type T7 RNA polymerase.

FIG. 10: Dot blot hybridization diagrams of dsRNA products of T7 RNA polymerase mutants Mut-231, Mut-246, Mut-258, and Mut-271. WT represents wild-type T7 RNA polymerase.

FIG. 11: Shows a graph of quantitation results of dsRNA from 4 k DNA templates. WT represents wild-type T7 RNA polymerase.

FIG. 12: Shows a graph of quantitation results of dsRNA from 8 k DNA templates. WT represents wild-type T7 RNA polymerase.

FIG. 13: Purity test results of the mutant 4 k DNA template. WT represents wild-type T7 RNA polymerase. RFU refers to relative fluorescence units and nt refers to nucleotides.

FIG. 14: Purity test results of the mutant 8 k DNA template. WT represents wild-type T7 RNA polymerase. RFU refers to relative fluorescence units and bp refers to base pairs.

FIG. 15: The yield of GMP-grade co-transcriptional products for T7 RNA polymerase mutants Mut-029, Mut-057, Mut-119, Mut-258, and WT with the addition of different concentrations (i.e., 0 mM, 2.5 mM, 5.0 mM, or 10.0 mM) of cap analog m7G(5′)ppp(5′)(2′OMeA)pU. WT represents wild-type T7 RNA polymerase.

### DETAILED DESCRIPTION

The present disclosure provides a mutant T7 RNA polymerase protein comprising a substitution at one or at least two of the amino acid residues of a wild-type T7 RNA polymerase comprising the amino acid sequence set forth in SEQ ID NO:1 or a T7 RNA polymerase protein having at least 90% sequence identity to a mutant T7 RNA polymerase of this disclosure, compositions comprising a mutant T7 RNA polymerase protein of this disclosure, transcription kits comprising a mutant T7 RNA polymerase protein of this disclosure, and methods of generating a transcription product or a capped transcription product using a mutant T7 RNA polymerase protein of this disclosure.

### Definitions

The term “herein” means the entire application.

Unless otherwise defined herein, scientific and technical terms used in this application shall have the meanings that are commonly understood by those of ordinary skill in the art. In case of conflict, the present specification, including definitions, will control.

It should be understood that any of the embodiments described herein, including those described under different aspects of the disclosure and different parts of the specification can be combined with one or more other embodiments of this disclosure, unless explicitly disclaimed or improper. Combination of embodiments are not limited to those specific combinations recited in the various multiple dependent embodiments herein.

All of the publications, patents and published patent applications referred to in this application are specifically incorporated by reference herein. In case of conflict, the present specification, including its specific definitions, will control.

Throughout this specification and embodiments, the word “comprise” or variations such as “comprises” or “comprising” will be understood to imply the inclusion of a stated integer (or components) or group of integers (or components), but not the exclusion of any other integer (or components) or group of integers (or components).

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article.

In order that the disclosure may be more readily understood, certain terms are first defined. These definitions should be read in light of the remainder of the disclosure as understood by a person of ordinary skill in the art. Unless defined otherwise, all technical and scientific terms used herein have the same meaning as commonly understood by a person of ordinary skill in the art. Additional definitions are set forth throughout the detailed description.

The term “identity” refers sequence identity that may be calculated using a substitution matrix or other known methods in the art. The sequence identity may be calculated using, e.g., BLOSUM62 matrix and methods described in Henikoff et al., PNAS, 89(22):10915-10919 (1992). In some embodiments, the sequence identity of a mutant T7 RNA polymerase protein is at least 90% sequence identity to the amino acid sequence of a naturally occurring wild-type T7 RNA polymerase. In some embodiments, the sequence identity of a mutant T7 RNA polymerase protein at least 90%, at least 91%, at least 92%, at least 93%, at least 94%, at least 95%, at least 96%, at least 97%, at least 98%, at least 99%, at least 99.5%, at least 99.8%, or at least 99.9% sequence identity to the amino acid sequence of a naturally occurring wild-type T7 RNA polymerase.

The terms “mutant T7 RNA polymerase protein,” “mutant T7 RNA polymerase proteins,” “T7 RNA polymerase mutant,” or “T7 RNA polymerase mutants” refer to proteins composed of L-amino acids or achiral amino acids (e.g., achiral glycine).

The term “activity and enzymatic properties” refers to the transcriptional activity of the mutant T7 RNA polymerase protein to generate a transcription product (e.g., RNA) or a capped transcription product (e.g., capped RNA) and to produce during transcription one or more of a lower amount of dsRNA by-products, a higher product integrity, a higher yield, a higher transcriptional activity, or a lower cap analog usage as compared to a wild-type T7 RNA polymerase.

The term “substantially the same” or “substantially affect” in the context of the activity and enzymatic properties of a mutant T7 RNA polymerase protein refers to a deviation of a measured testing value that does not exceed 20% under the same testing conditions. In some embodiments, testing comprise measuring transcriptional activity, measuring enzyme activity in a transcription reaction, measuring volume activity in a transcription reaction, measuring specific activity in a transcription reaction, measuring amount of dsRNA by-products, measuring product integrity, measuring product yield, or measuring cap analog usage.

The term “pharmaceutically acceptable additives” refers to one or more additives including, but not limited to, a buffer, a saccharide, a stabilizer, a cryoprotectant, a lyoprotectant, and a chelating agent. During transcription to generate RNA or capped RNA, the resulting RNA or capped RNA can either be naked or formulated in a suitable form for delivery to a subject, e.g., a human. Formulations or “pharmaceutical dosage forms” can include, e.g., liquid formulations (solutions, suspensions, dispersions), topical formulations (gels, ointments, drops, creams), or liposomal formulations. In some embodiments, the buffer is selected from the group of 4-(2-hydroxyethyl)-1-piperazineethanesulfonic acid (HEPES), an acetate buffer, an acetate buffer analogue, a phosphoric acid buffer, a phosphate buffer, a citric acid buffer, and a citrate buffer. In some embodiments, the lyoprotectant is a saccharide. In some embodiments, the saccharide is selected from the group of monosaccharides, disaccharides, trisaccharides, oligosaccharides, and polysaccharides. In some embodiments, the saccharide is selected from glucose, trehalose, and saccharose. In some embodiments, the cryoprotectant is a glycol. In some embodiments, the cryoprotectant is selected from the group of ethylene glycol, propylene glycol, and glycerol. In some embodiments, the chelating agent comprises EDTA.

### Compositions

In one aspect, the present disclosure provides a mutant T7 RNA polymerase protein selected from the group of:
a1: a protein comprising a substitution at one or at least two of the amino acid residues of a wild-type T7 RNA polymerase comprising the amino acid sequence set forth in SEQ ID NO:1, the amino acid residues being selected from the group of:

### I6, I19, K60, N67, A70, N86, F162, V186, N233, A247, P277, A465, L651, or N764; or

a2: a protein comprising an amino acid sequence having at least 90% sequence identity to a mutant T7 RNA polymerase protein of a1, and whose activity and enzymatic properties are substantially the same as one or more of a mutant T7 RNA polymerase protein of a1.

In some embodiments, the mutant T7 RNA polymerase protein of this disclosure further comprises a substitution at one or at least two of the amino acid residues selected from the group of F11, I82, D87, K180, V214, M369, Y457, H523, K610, A615, 5686, or K740.

In some embodiments, the mutant T7 RNA polymerase protein of this disclosure comprises one or a combination of at least two of an amino acid substitution selected from the group of: I6G, F11L, I19T, K60I, N67S, A70Q, A70T, I82V, N86D, D87G, F162S, K180E, K180D, V186I, V214A, N233D, A247T, A247I, P277L, M369S, M369T, Y457H, A465T, H523R, K610R, A615T, L651M, S686G, K740R, or N764D.

In some embodiments, the mutant T7 RNA polymerase protein of this disclosure comprises one or a combination of at least two of an amino acid substitution selected from the group of: I6G, F11L, I19T, K60I, N67S, A70Q, N86D, F162S, K180E, K180D, V186I, V214A, N233D, A247I, P277L, M369S, Y457H, A465T, H523R, L651M, or N764D.

In another aspect, the present disclosure provides a mutant T7 RNA polymerase protein selected from the group of:
b1: a protein comprising a substitution at one or at least two of the amino acid residues of a wild-type T7 RNA polymerase comprising the amino acid sequence set forth in SEQ ID NO:1, the amino acid residues being selected from the group of:

### I6G, F11L, I19T, K60I, N67S, A70Q, A70T, N86D, F162S, K180E, K180D, V186I, V214A, N233D, A247T, A247I, P277L, M369S, Y457H, A465T, H523R, L651M, or N764D;

b2: a protein comprising at least two of the amino acid residues of a wild-type T7 RNA polymerase comprising the amino acid sequence set forth in SEQ ID NO:1, the amino acid residues being selected from the group of:

### I82V, D87G, M369T, K610R, A615T, S686G, or K740R;

b3: the protein of b1 further comprising a substitution at one or at least two of the amino acid residues of a wild-type T7 RNA polymerase comprising the amino acid sequence set forth in SEQ ID NO:1, the amino acid residues being selected from the group of:

### I82V, D87G, M369T, K610R, A615T, S686G, or K740R; or

b4: a protein comprising an amino acid sequence having at least 90% sequence identity to a mutant T7 RNA polymerase protein of b1, b2, or b3, and whose enzymatic activity and properties are substantially the same as one or more of a mutant T7 RNA polymerase protein of b1, b2, or b3.

In some embodiments, the mutant T7 RNA polymerase protein of this disclosure (as compared to the corresponding protein of Paragraphs [0063]-[0067]) further comprises one or more of an additional substitution, deletion, or addition of an amino acid residue, the protein having substantially the same activity and enzymatic properties as one or more of a mutant T7 RNA polymerase protein of Paragraphs [0063]-[0067].

In another aspect, the present disclosure provides a mutant T7 RNA polymerase protein selected from the group of:
c1: a protein comprising a substitution at one or at least two of the amino acid residues of a wild-type T7 RNA polymerase comprising the amino acid sequence set forth in SEQ ID NO:1, wherein the substitution is selected from the group of:
(1) N86D/A615T;
(2) F162S;
(3) F162S/K180E;
(4) A247I;
(5) P277L;
(6) A70Q/F162S/K180E;
(7) N233D/A465T;
(8) D87G/N233D/V186I;
(9) A70T/P277L;
(10) N67S/I82V/Y457H/K610R/L651M/N764D;
(11) F162S/A247T;
(12) F162S/N233D/A247T;
(13) V214A;
(14) I19T/K180D/M369T;
(15) F162S/K180E/A247I;
(16) A70Q/V214A;
(17) A465T;
(18) F11L;
(19) K180E;
(20) M369S/S686G;
(21) K60I/A465T/K740R;
(22) H523R;
(23) A70T/K180D;
(24) I6G;
(25) A70Q/K180E;
(26) A70Q;
(27) V214A/A465T; or
(28) A70Q/A247T; or
c2: a protein comprising an amino acid sequence having at least 90% sequence identity to a mutant T7 RNA polymerase protein of c1, and whose activity and enzymatic properties are substantially the same as one or more of a mutant T7 RNA polymerase protein of c1.

In some embodiments, the mutant T7 RNA polymerase protein of this disclosure (as compared to the corresponding protein of Paragraph [0069]) further comprises one or more of an additional substitution, deletion, or addition of an amino acid residue, the protein having substantially the same activity and enzymatic function as one or more of a mutant T7 RNA polymerase protein of Paragraph [0069].

In some embodiments, the mutant T7 RNA polymerase protein of this disclosure further comprises one or more of a tag or an enzyme cleavage site at one or both of the N-terminus or the C-terminus of the protein, and wherein the tag or the enzyme cleavage site does not substantially affect the activity and enzymatic properties of the mutant T7 RNA polymerase protein. In some embodiments, the tag is a His tag. In some embodiments, the enzyme cleavage site is a thrombin cleavage site. In some embodiments, the one or more of the His tag or the thrombin cleavage site is at the N-terminus of the protein.

In some embodiments, the mutant T7 RNA polymerase protein of this disclosure as compared to a wild-type T7 RNA polymerase comprising the amino acid sequence set forth in SEQ ID NO:1, is characterized by producing during transcription one or more of a lower amount of dsRNA by-products, a higher product integrity, a higher yield, a higher transcriptional activity, or a lower cap analog usage.

In some embodiments, the mutant T7 RNA polymerase protein of this disclosure is characterized by being capable of catalyzing co transcriptional capping.

In another aspect, the present disclosure provides a composition comprising the mutant T7 RNA polymerase protein of this disclosure, and one or more of an in vivo transcription reagent or in vitro transcription reagent.

### Transcription Kits

In another aspect, the present disclosure provides a transcription kit comprising a mutant T7 RNA polymerase protein of this disclosure or a composition of this disclosure. In some embodiments, the transcription kit is a co-transcription capping kit.

In some embodiments, the transcription kit of this disclosure further comprises a cap analog and a buffer system. In some embodiments, the buffer system comprises: 30-50 mM Tris, 5-20 mM DTT, 1-5 mM spermidine, and 20-100 mM MgCl₂.

### Methods of Generating a Transcription Product or Capped Transcription Product

In another aspect, the present disclosure provides a method of generating a transcription product of a deoxyribonucleic acid (DNA) comprising contacting the DNA with a mutant T7 RNA polymerase protein of this disclosure or a composition of this disclosure.

In some embodiments, the method of this disclosure further comprises contacting the transcription product with a capping enzyme to form a capped transcription product.

In another aspect, the present disclosure provides a method of generating a capped transcription product of a deoxyribonucleic acid (DNA) comprising:
(i) mixing the DNA, a cap analog, and a mutant T7 RNA polymerase protein of this disclosure, or a composition of this disclosure, in a buffer system; and
(ii) generating the capped transcription product through transcription of the DNA using the mixture of step (i).

In some embodiments, the methods of this disclosure further comprise combining the transcription product or the capped transcription product with a pharmaceutically acceptable additive to produce a pharmaceutical dosage form.

### EXAMPLES

The examples of the present disclosure are further described below in conjunction with the accompanying drawings, but the description of the examples does not limit the scope of protection of the present disclosure in any way.

Unless otherwise defined, all technical and scientific terms used herein have the same meanings as commonly understood by those skilled in the technical field of the present disclosure. The terms used in the specification of the present disclosure are intended solely for describing specific embodiments and not intended to limit the scope of the present disclosure.

The substances or instruments used in the following examples can be obtained from conventional commercial sources unless otherwise specified.

### Example 1

1. The following mutants (Table 1) were obtained through directed evolution. Using methods such as homologous recombination, enzyme ligation, and sequence synthesis, the DNA sequences encoding the T7 RNA polymerase mutants, N-terminus 6*His tag, and thrombin cleavage site were constructed into the pAR1219 vector (pAR1219 purchased from Sigma-Aldrich, Cat #T2076), then transformed into Escherichia coli BL21. The sequence of the wild-type T7 RNA polymerase is shown in SEQ ID NO: 1, and its corresponding nucleotide sequence is shown in SEQ ID NO: 2.

TABLE 1

| Mutant Number | Mutation Site (Based on SEQ ID NO: 1) |
|---|---|
| Mut-022 | N86D/A615T |
| Mut-025 | F162S |
| Mut-029 | F162S/K180E |
| Mut-049 | A247I |
| Mut-050 | P277L |
| Mut-057 | A70Q/F162S/K180E |
| Mut-077 | N233D/A465T |
| Mut-082 | D87G/N233D/V186I |
| Mut-083 | A70T/P277L |
| Mut-103 | N67S/182V/Y457H/K610R/L651M/N764D |
| Mut-119 | F162S/A247T |
| Mut-134 | F162S/N233D/A247T |
| Mut-139 | V214A |
| Mut-144 | I19T/K180D/M369T |
| Mut-161 | F162S/K180E/A247I |
| Mut-162 | A70Q/V214A |
| Mut-168 | A465T |
| Mut-176 | F11L |
| Mut-180 | K180E |
| Mut-184 | M369S/S686G |
| Mut-195 | K60I/A465T/K740R |
| Mut-197 | H523R |
| Mut-207 | A70T/K180D |
| Mut-218 | I6G |
| Mut-231 | A70Q/K180E |
| Mut-246 | A70Q |
| Mut-258 | V214A/A465T |
| Mut-271 | A70Q/A247T |

2. The mutants with sequences verified to be correct were inoculated into 4 mL of fresh LB liquid culture medium, and cultured overnight at 37° C. and 200 rpm on a shaker;
3. Each mutant bacterial solution was inoculated into three 800 mL portions of fresh 2×YT liquid culture medium at a 1% ratio, cultured at 37° C. and 250 rpm until an OD₆₀₀ value of approximately 0.6 was reached, then added with 0.5 mM IPTG and cultured at 16° C. and 250 rpm for approximately 16 hours to induce the expression of exogenous proteins;
4. The bacterial solution of each mutant was collected and centrifuged at 4° C. and 5,000 rpm for 10 minutes, and the supernatant was discarded;
5. Approximately 4 g of wet bacteria was weighed out from each and dissolved in approximately 40 mL of 20 mM Tris-HCl buffer at pH 8.0, and the remaining bacteria was stored in a refrigerator at −20° C.;
6. The ultrasonic crusher was cleaned and ultrasonic crushing of the mutants was performed in batches at 60% power, using 3 s ON/5 s OFF cycles, for approximately 30 minutes, until the solution became mostly clear;
7. The ultrasonic crushing solution was centrifuged at 4° C. and 15,000 rpm for 30 minutes, and the supernatant was collected;
8. The AKTA Pure-150 device was debugged and the mutants were purified using a nickel column with a column volume of 10 mL. The purification steps and purification buffer are as shown in Table 2 below:

TABLE 2

| | | Flow Rate | |
|---|---|---|---|
| Step | Buffer | (mL/min) | Volume (mL) |
| Balance | Buffer A | 5 | 50 |
| Sample loading | Buffer A | 5 | 40 |
| Washing 1 | Buffer A | 5 | 50 |
| Washing 2 | 20% Buffer B | 5 | 50 |
| Elution | 60% Buffer B | 5 | 40 |
| Washing 3 | 100% Buffer B | 5 | 100 |

Buffer A formulation: 20 mM Tris-HCl, 100 mM NaCl, 10% glycerol (pH 8.0);
Buffer B formulation: 20 mM Tris-HCl, 100 mM NaCl, 10% glycerol, 500 mM imidazole (pH 8.0);

9. The eluent was collected and dialyzed into 1 μL of Buffer C, followed by the debugging of the AKTA Pure-150 device, and the mutants were purified using a DEAE column with a column volume of 5 mL, with the purification steps and purification buffer as shown in Table 3 below:

TABLE 3

| | | Flow Rate | Volume |
|---|---|---|---|
| Step | Buffer | (mL/min) | (mL) |
| Balance | Buffer C | 5 | 50 |
| Sample | Buffer C | 5 | 1,000 |
| loading | | | |
| Washing | Buffer C | 5 | 100 |
| 1 | | | |
| Washing | 10% Buffer D | 5 | 50 |
| 2 | | | |
| Elution | 30% Buffer D | 5 | 20 |
| Washing | 100% Buffer D | 5 | 50 |
| 3 | | | |

Buffer C formulation: 20 mM Tris-HCl, 10% glycerol, 1 mM EDTA, 2 mM DTT (pH 7.5);
Buffer D formulation: 20 mM Tris-HCl, 10% glycerol, 1 mM EDTA, 2 mM DTT, 1M NaCl (pH 7.5);

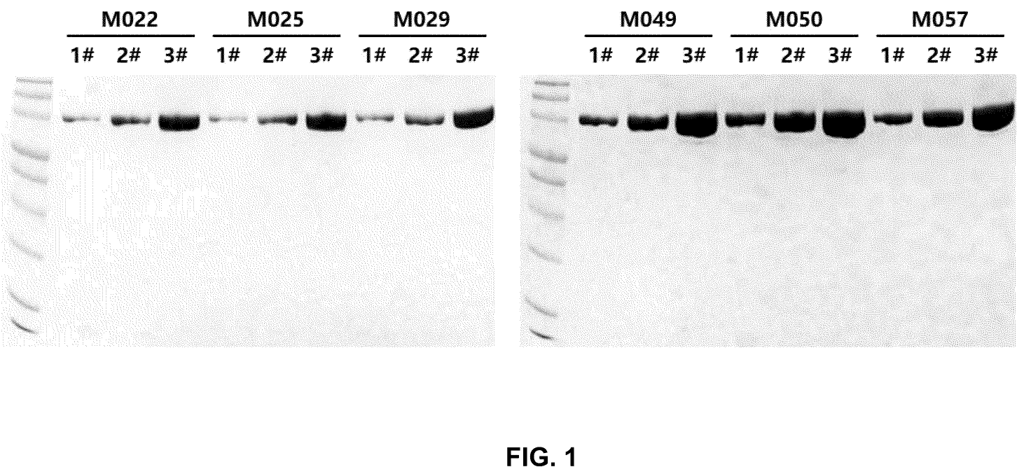

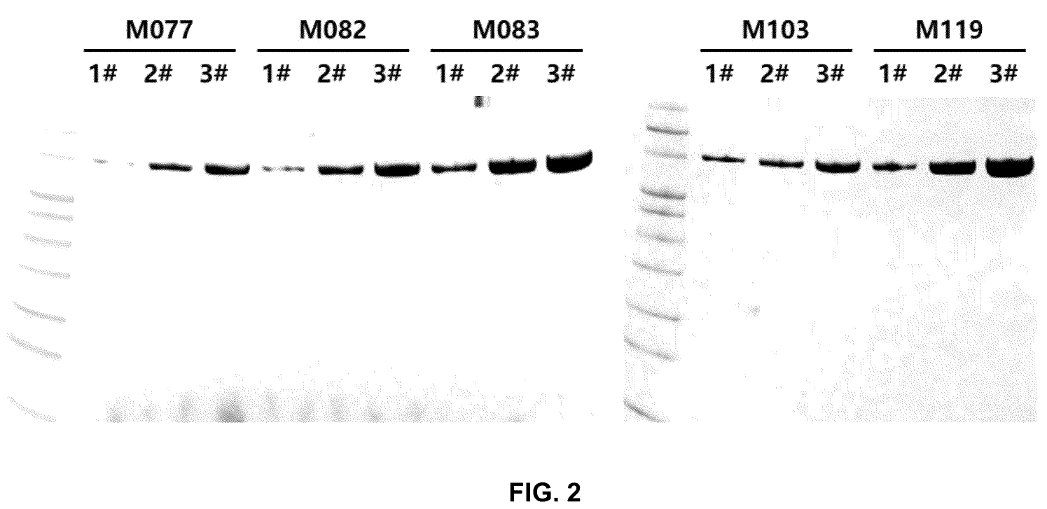

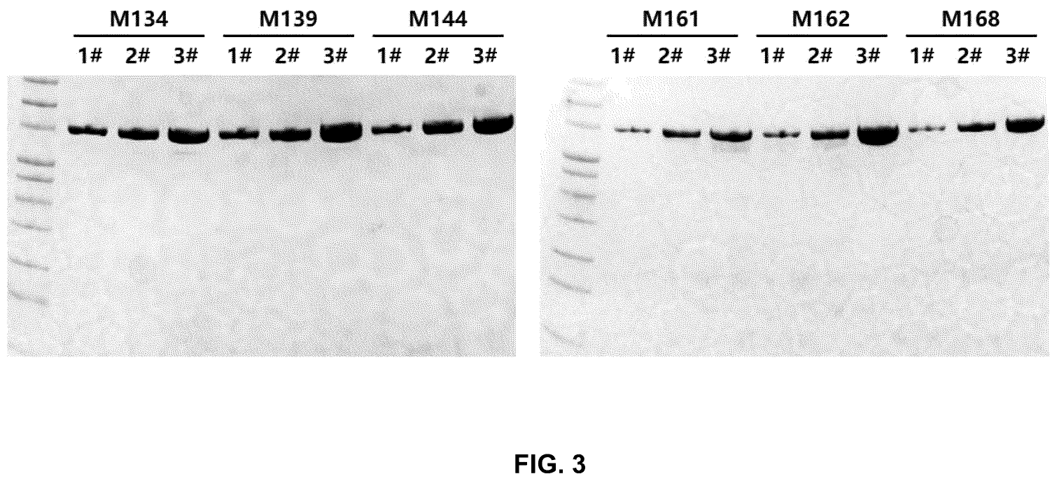

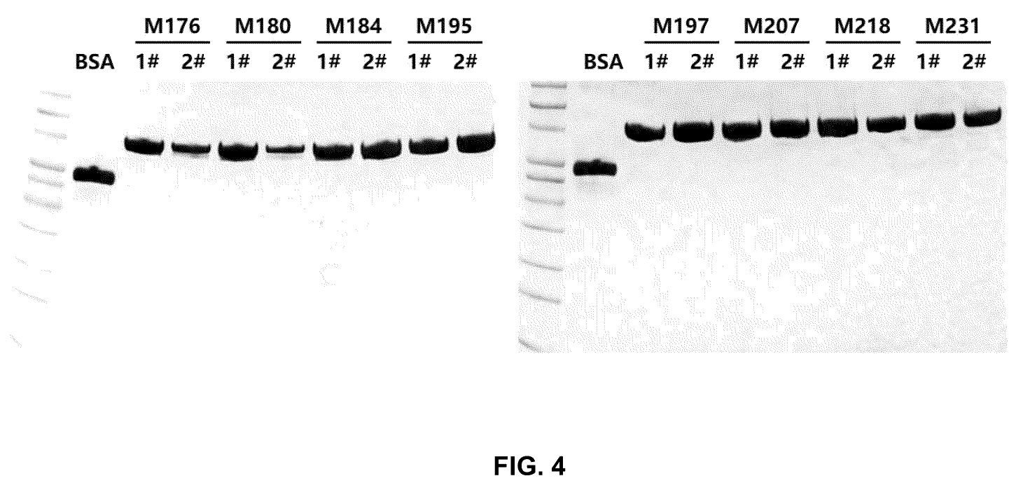

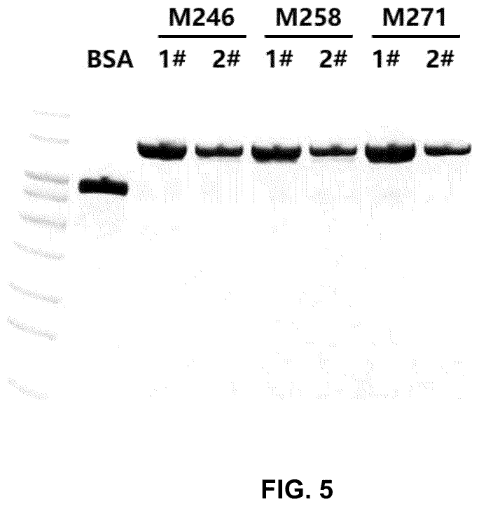

10. The eluent was collected, supplemented with 50% volume of glycerol and mixed thoroughly, then filtered through a 0.22 m membrane filter, and electrophoresis was then performed to assess the purity of each mutant and estimate the protein concentration. As shown in FIGS. 1-5, the purity of each mutant exceeded 90%, meeting the requirements for subsequent characterization.

### Example 2

1. Each mutant protein was diluted to approximately 0.5 mg/mL, then further gradient diluted to approximately 0.4 mg/mL, 0.2 mg/mL, 0.1 mg/mL, and 0.05 mg/mL;
2. The T7 RNA polymerase from Yeasen Biotechnology Commercialization Reagent Kit 10623ES60 was used for the gradient dilution of the enzyme, starting from 250 U/μL to concentrations of 100 U/μL, 50 U/μL, 25 U/μL, 10 U/μL, 5 U/μL, 2 U/μL, and 1 U/μL, for later use;
3. Following the components and reaction conditions of the reagent kit, in vitro transcription reactions were performed individually on the aforementioned enzymes, with the transcription template being the 4K template shown in SEQ TD NO: 3;
4. The working solution was prepared according to the requirements of the AAT Bioquest Pyrophosphate Assay Kit AAT-21611, with each gradient mutant and the commercial T7 RNA polymerase gradient dilution sample mixed with the pyrophosphate quantitative working solution, followed by incubation at room temperature for 20 minutes, and the fluorescence value was then determined using a microplate reader under the conditions of Ex/Em=316 nm/456 nm (cutoff 420 nm);
5. A standard curve was plotted with the enzyme activity of the diluted commercial T7 RNA polymerase against the fluorescence signal, and the enzyme activity of the mutant after dilution was calculated based on the signal values of the mutants. The volume activity and specific activity of each mutant are as follows in Table 4:

TABLE 4

| | | | Specific |
|---|---|---|---|
| Mutant | Volume Activity | Protein Concentration | Activity |
| Number | (U/μL) | (mg/mL) | (U/μg) |
| WT | 654.3 | 1.73 | 378.21 |
| Mut-022 | 2,641.0 | 5.34 | 494.20 |
| Mut-025 | 700.0 | 1.33 | 524.74 |
| Mut-029 | 495.0 | 0.87 | 568.97 |
| Mut-049 | 444.6 | 1.25 | 356.82 |
| Mut-050 | 518.3 | 1.23 | 422.76 |
| Mut-057 | 490.6 | 1.31 | 375.36 |
| Mut-077 | 754.9 | 1.77 | 427.10 |
| Mut-082 | 1,063.5 | 2.65 | 401.70 |
| Mut-083 | 777.5 | 1.43 | 542.28 |
| Mut-103 | 990.0 | 2.01 | 492.29 |
| Mut-119 | 418.0 | 1.11 | 376.29 |
| Mut-134 | 318.0 | 1.03 | 310.24 |
| Mut-139 | 732.0 | 1.62 | 451.85 |
| Mut-144 | 396.1 | 1.24 | 318.37 |
| Mut-161 | 413.6 | 1.44 | 286.89 |
| Mut-162 | 713.5 | 1.66 | 430.25 |
| Mut-168 | 645.1 | 1.98 | 325.81 |
| Mut-176 | 1,401.0 | 3.26 | 429.75 |
| Mut-180 | 1,401.0 | 3.26 | 429.75 |
| Mut-184 | 317.9 | 0.56 | 567.61 |
| Mut-195 | 457.8 | 0.92 | 497.64 |
| Mut-197 | 995.4 | 1.92 | 518.46 |
| Mut-207 | 1,555.0 | 1.98 | 785.34 |
| Mut-218 | 448.0 | 1.12 | 400.00 |
| Mut-231 | 492.0 | 1.14 | 431.58 |
| Mut-246 | 940.0 | 1.66 | 566.27 |
| Mut-258 | 628.8 | 1.27 | 495.13 |
| Mut-271 | 561.5 | 1.41 | 398.25 |

The specific activity of the mutants obtained was comparable to or higher than that of the wild-type T7 RNA polymerase. Among them, mutant Mut-207 exhibited the highest specific activity, which was approximately twice that of the wild-type T7 RNA polymerase.

### Example 3

1. The in vitro transcription system for T7 RNA polymerase was prepared according to the following table (Table 5). The wild-type T7 RNA polymerase or mutant was diluted to 250 U/μL, or the corresponding volume was added based on enzyme activity conversion, and allowed to react at 37° C. for 3 hours.

TABLE 5

| | | | Final |
|---|---|---|---|
| Component | Volume | | Concentration |
| 10× Transcription Buffer (Mg²⁺ | 2.0 | μL | 1× |
| Plus) | | | |
| CTP/GTP/ATP/UTP (100 mM each) | 0.4 μL each | | 2 mM each |
| T7 RNA Polymerase | 250 | U | — |
| RNase inhibitor (40 U/μL) | 40 | U | — |
| RNase-free H₂O | Make up to 18 μL | | — |
| Template DNA | 500 | ng | — |

The 4 template was the in vitro transcription template use or activity measurement (SEQ ID NO: 3). The RNase inhibitor is a product from Yeasen Biotechnology, Cat #10603, and the CTP/GTP/ATP/UTP are products from Yeasen Biotechnology, Cat #10133;
2. 115 μL of RNase-free H₂O and 15 μL of 3M sodium acetate (pH 5.2) were added to 20 μL of the reaction mixture and mixed well;
3. An equal volume of phenol-chloroform (1:1) was used to perform one extraction, followed by two extractions with an equal volume of chloroform, and the supernatant was then collected and transferred to a new RNase-free EP tube;
4. Twice the volume of anhydrous ethanol is added to precipitate RNA. After mixing well, the mixture is left at −20° C. for at least 30 minutes, then centrifuged at 4° C. and 16,500 rpm for 15 minutes, and the precipitate was then collected;
5. 500 μL of ice-cold 70% ethanol was added to wash the RNA precipitate twice;
6. The RNA precipitate was dissolved in 20 μL of RNase-free H₂O;
7. The transcription products of the wild-type T7 RNA polymerase and each mutant were separately diluted 50-fold and 100-fold, ensuring that the RNA concentration did not exceed the standard curve range of the Nanodrop. The RNA concentration of each mutant under the two dilution conditions was measured. If the values obtained for the 50-fold and 100-fold dilutions were not significantly different when multiplied by their respective dilution factors, the average was taken as the mean value of the mutant's transcription product. If the difference between the two dilution factors was significant, the RNA concentration needed to be remeasured. The results of the RNA yield for the wild-type and each mutant are in Table 6:

TABLE 6

| | | Percentage |
|---|---|---|
| Mutant Number | Yield (mg/mL) | (%) |
| WT | 10.28 | — |
| Mut-022 | 10.73 | 104% |
| Mut-025 | 9.86 | 96% |
| Mut-029 | 10.66 | 104% |
| Mut-049 | 10.65 | 104% |
| Mut-050 | 10.29 | 100% |
| Mut-057 | 9.93 | 97% |
| Mut-077 | 10.49 | 102% |
| Mut-082 | 10.13 | 99% |
| Mut-083 | 10.44 | 102% |
| Mut-103 | 10.39 | 101% |
| Mut-119 | 10.10 | 98% |
| Mut-134 | 10.33 | 100% |
| Mut-139 | 10.04 | 98% |
| Mut-144 | 10.29 | 100% |
| Mut-161 | 10.05 | 98% |
| Mut-162 | 9.88 | 96% |
| Mut-168 | 9.93 | 97% |
| Mut-176 | 10.01 | 97% |
| Mut-180 | 10.12 | 98% |
| Mut-184 | 9.70 | 94% |
| Mut-195 | 9.83 | 96% |
| Mut-197 | 9.86 | 96% |
| Mut-207 | 10.67 | 104% |
| Mut-218 | 10.99 | 107% |
| Mut-231 | 10.01 | 97% |
| Mut-246 | 9.85 | 96% |
| Mut-258 | 10.55 | 103% |
| Mut-271 | 10.22 | 99% |

### Example 4

1. An appropriate amount of RNA product was placed into a new RNase-free EP tube, diluted accurately to 1,200 ng/μL using RNase-free H₂O, and mixed well. To prevent RNA degradation, the dilution process was performed on ice;
2. After brief centrifugation, the diluted RNA sample was heated at 95° C. for 3 minutes and immediately placed on ice to ensure that the RNA secondary structure was unfolded;
3. The nitrocellulose (NC) membrane was cut to an appropriate size, and 2 μL of each mutant product was sequentially pipetted onto the NC membrane, with 2-4 drops for each sample. During the spotting process, care was taken not to touch the NC membrane with the pipette tip and the sample was allowed to diffuse freely. After the membrane was partially dried, the next sample was dispensed;
4. After spotting, the NC membrane was transferred to a 37° C. oven for 30 minutes of processing to crosslink RNA with the membrane;
5. The NC membrane was removed and transferred to a clean plastic box, then washed with 10 mL of Tris-Buffered Saline-Tween (TBST) for 5 minutes to remove unbound RNA;
6. The TBST supernatant was discarded, then 10 mL of TBST with blocking buffer (QuickBlock™ Blocking Buffer, Beyotime P0231) was added, followed by incubation at room temperature at 30 rpm for 1 hour;
7. The blocking buffer was discarded, then blocking buffer containing 1% mouse anti-double-stranded RNA (J2) antibody (10010500-Nordic-Mubio) was added, followed by incubation overnight at 4° C.;
8. The primary antibody solution was removed and the NC membrane was washed with 10 mL of TBST solution, followed by incubation at room temperature at 30 rpm for 10 minutes, repeated three times;
9. 10 mL of TBST containing 1% goat anti-mouse antibody (Peroxidase AffiniPure Goat Anti-Mouse IgG(H+L), Yeasen Biotechnology product, Cat #33201) was added, followed by incubation at room temperature at 30 rpm for 1 hour;
10. Step 8 was repeated to wash the NC membrane;
11. Color development was performed according to the instruction manual of the BCIP/NBT color reagent kit (Solarbio, PR1100), and qualitative analysis of the dsRNA content in the products of different mutants was conducted.

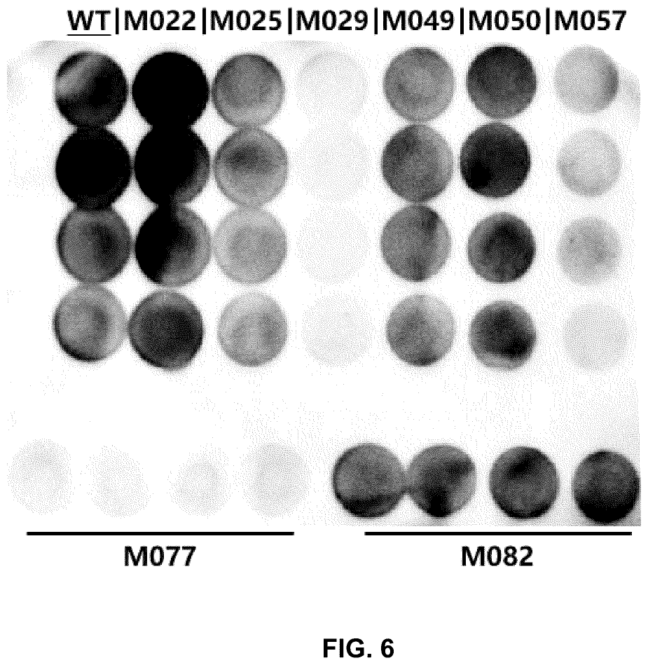

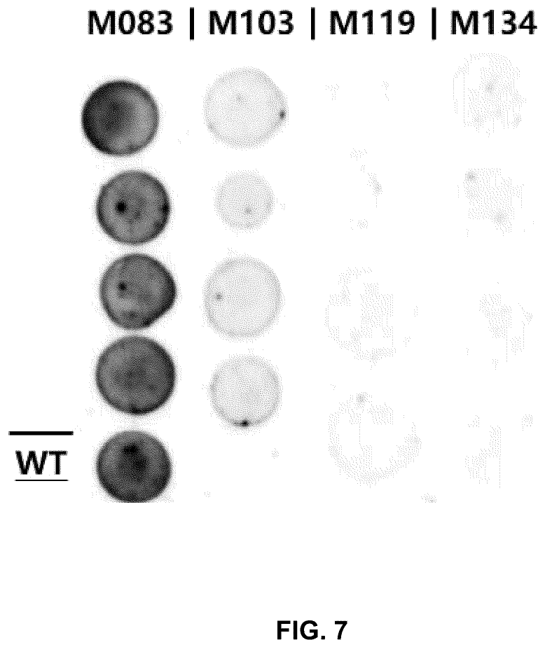

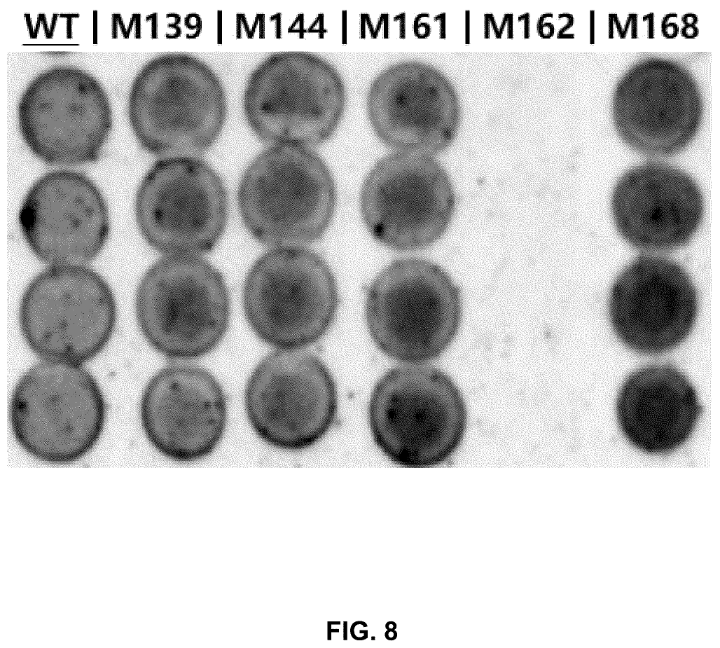

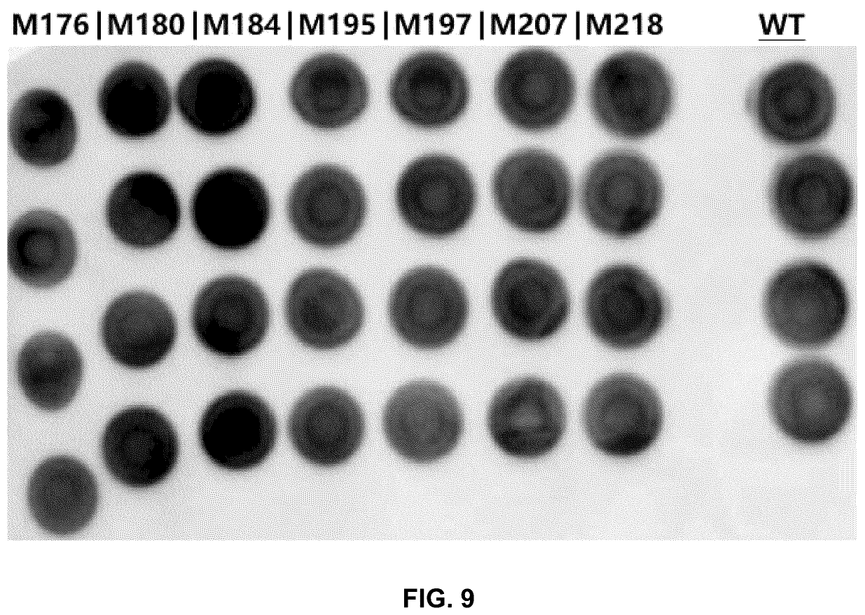

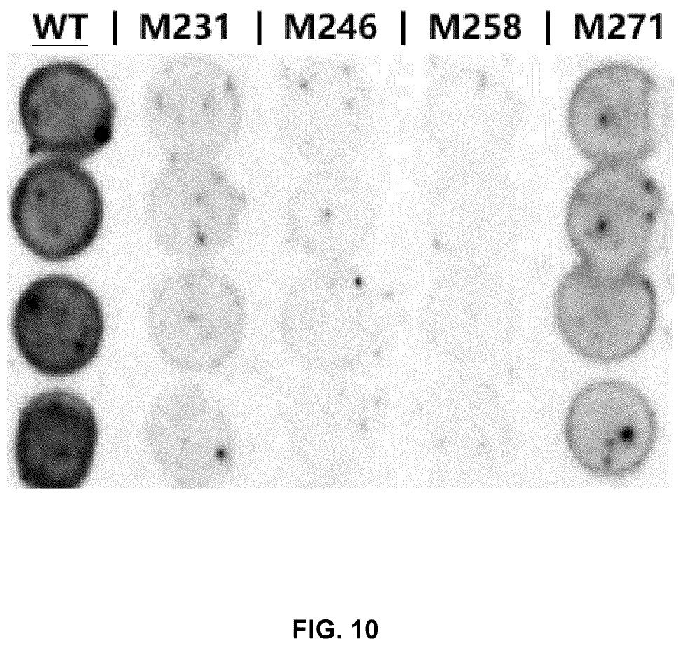

As shown in FIG. 6-10, the grayscale intensities of the mutant spots for Mut-025, Mut-029, Mut-049, Mut-057, Mut-077, Mut-103, Mut-119, Mut-134, Mut-162, Mut-231, Mut-246, Mut-258, and Mut-271 on the NC membrane were significantly lower than that of the wild type T7 RNA polymerase. The grayscale intensities of the mutant spots for Mut-022, Mut-050, Mut-081, Mut-083, Mut-139, Mut-144, Mut-161, Mut-168, Mut-176, Mut-180, Mut-184, Mut-195, Mut-197, Mut-207, and Mut-218 were comparable to that of the wild type, and quantitative analysis was required. According to the principles of the reagent kit, the higher the grayscale value of the mutant product after dot-blot hybridization, the higher the dsRNA content.

### Example 5

1. Referencing Example 3, in vitro transcription was performed using the 8 k self-replicating template (SEQ ID NO: 4) and the RNA products were purified. At the same time, quantitative analysis of the in vitro transcription (IVT) products in the 4 k template transcription product in Example 3 was performed using a dsRNA quantitative reagent kit;
2. The procedures for the Yeasen Biotechnology dsRNA quantitative reagent kit (ELISA method, Cat #36717) were followed, and the STD1 standard sample was diluted gradually;
3. Each mutant and the diluted standard samples were added to an Elisa plate at 100 μL per well, with two replicate wells for each sample, ensuring that the sample loading was completed within 15 minutes. After sealing the plate with a sealing membrane, incubation was performed at room temperature at 500 rpm for 1 hour;
4. The liquid in the wells was discarded and the plate was washed five times with 1× wash buffer (250 μL/well), with the plate left to stand after each addition of the wash buffer, and the Elisa plate was then tapped dry;
5. The biotin-conjugated detection antibody that was pre-prepared was added to the working concentration to the Elisa plate at 100 μL/well, and incubated at room temperature at 500 rpm for 1 hour;
6. Step 4 was repeated;
7. Streptavidin-HRP that was pre-prepared was added to the Elisa plate at 100 μL/well, and incubated at room temperature at 500 rpm for 30 minutes;
8. Step 4 was repeated;
9. The substrate solution was allowed to return to room temperature for 10 minutes before use. The TMB substrate solution was then added to the Elisa plate at 100 μL/well, and incubated at room temperature for 30 minutes away from light;
10. The stop solution was added to the Elisa plate at 50 μL/well, and the Elisa plate was gently shaken to ensure uniform color development;
11. The absorbance values at wavelengths of 450 nm and 650 nm were immediately read (with 450 nm as the detection wavelength and 650 nm as the reference wavelength).

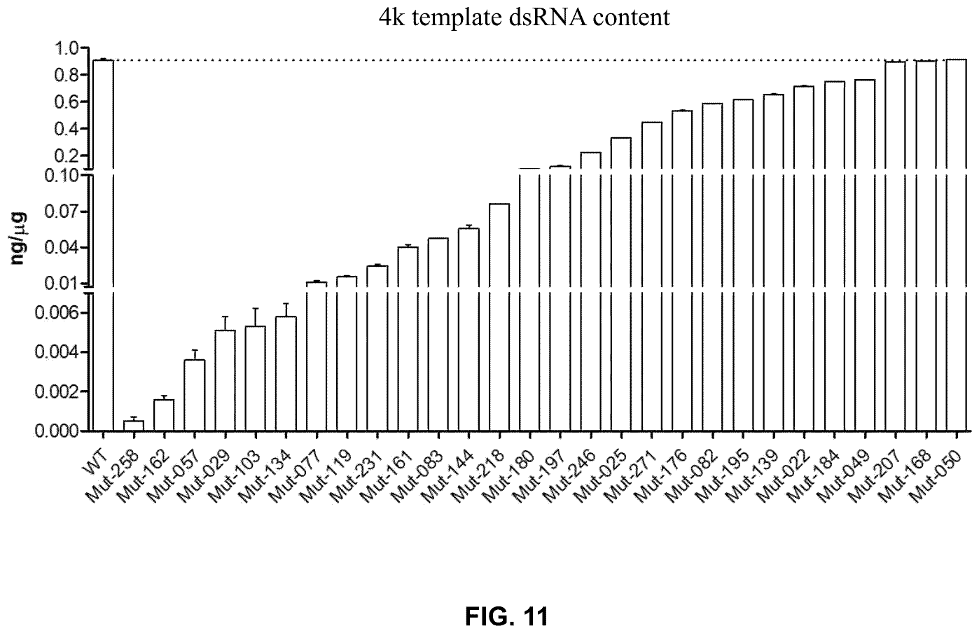

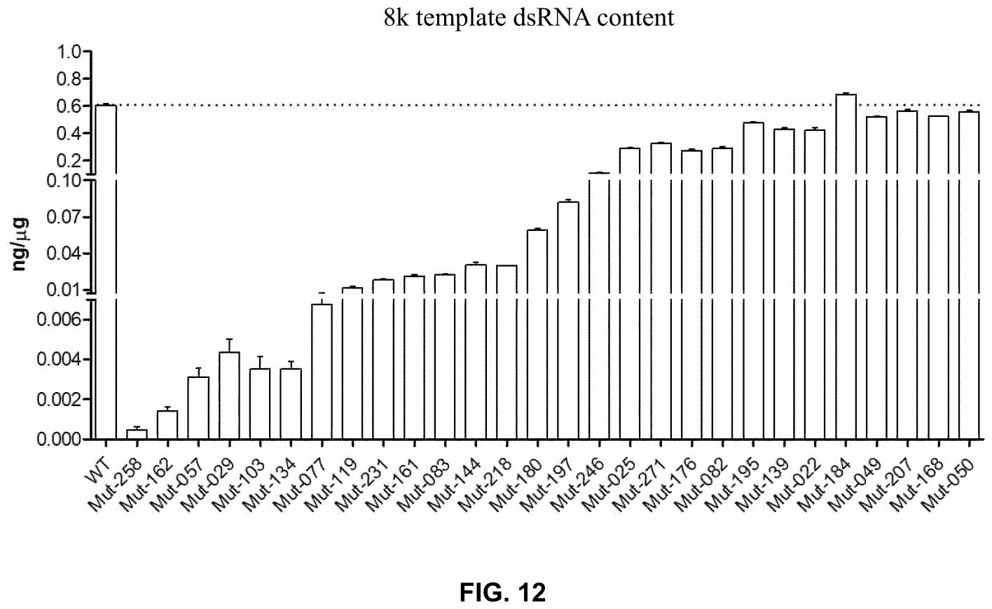

As shown in FIG. 11, the dsRNA content in the total RNA of the mutant for transcription from the 4 k template was significantly lower than that of the wild type (0.9070 ng/μg). Additionally, mutants Mut-029, Mut-057, Mut-103, Mut-134, and Mut-162 have dsRNA levels below 0.01 ng/μg total RNA (the dsRNA amount in 1 μg total RNA is lower than 0.01 ng), while the Mut-258 mutant has dsRNA levels below 0.001 ng/g total RNA. As shown in FIG. 12, for transcription from the 8 k template, mutants Mut-029, Mut-057, Mut-077, Mut-103, Mut-134, Mut-162, and Mut-258 exhibited dsRNA levels below 0.01 ng/μg total RNA. Specifically, the dsRNA level in Mut-258 was as low as 0.0003 ng/μg total RNA, indicating that the dsRNA yield was less than one millionth.

### Example 6

1. Referencing the relevant operations of Bioptic R1 RNA cartridge, prepare the diluent, Lower Marker, and other reagents. The aforementioned RNA samples are to 10 ng/μL;
2. The RNA samples were left at 70° C. for 2 minutes, and then placed on ice for more than 5 minutes;
3. The program parameters of the Qsep-100 device were set as needed;
4. The processed RNA samples were quickly added to the microplate and the program was ran;
5. The purity range of each sample was determined based on the recommended value range provided by the device.

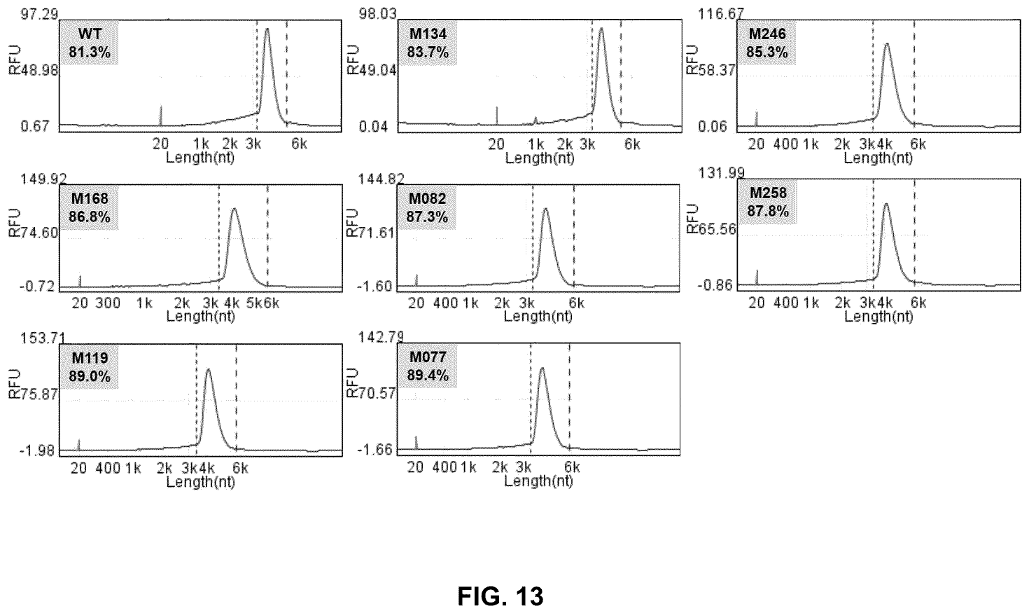

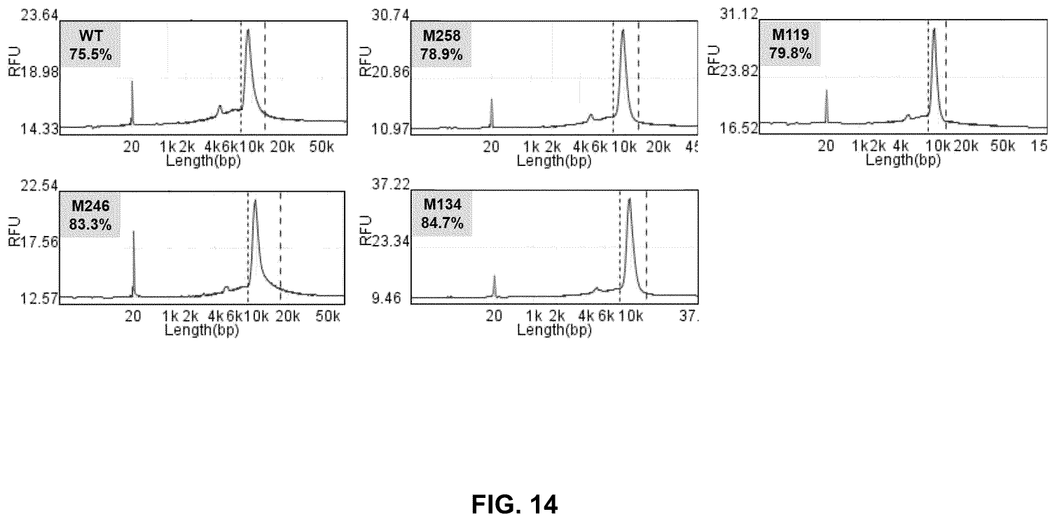

As shown in FIG. 13, for transcription from the 4 k template, the purity (i.e., integrity) of the products from T7 RNA polymerase mutants Mut-168, Mut-082, Mut-258, Mut-119, and Mut-077 increased by more than 5% compared to the wild type T7 RNA polymerase, of which the integrity of the Mut-077 product was capable of reaching up to 89.4%. As shown in FIG. 14, T7 RNA polymerase mutants Mut-258, Mut-119, Mut-246, and Mut-134 retained the advantage of increased product integrity when transcribing using the 8 k template, achieving a maximum transcription product integrity of approximately 85%, which was more than 9% integrity improvement compared to the wild type T7 RNA polymerase. The product integrity refers to the ratio of the number of full-length mRNAs to the total number of mRNAs in the transcription product.

### Example 7

1. The following reaction buffers was prepared: 40 mM Tris (pH=7.9±0.2), 10 mM DTT, 2 mM spermidine, and 46 mM MgCl₂ for in vitro co-transcription experiments of T7 RNA polymerase;
2. The cap analog m7G(5′)ppp(5′)(2′OMeA)pU mother solution was diluted with RNase free H₂O to an appropriate concentration for later use;
3. The in vitro transcription system was prepared according to the operation method indicated in Example 3. Four cap analog concentration gradients were configured for each mutant and wild-type T7 RNA polymerase, resulting in cap analog concentrations of 10 mM, 5 mM, 2.5 mM, and 0 mM in the final reaction system. The concentrations of other components in the in vitro transcription system were kept constant and they are made up to 20 μL with RNase-free H₂O in the system;
4. In vitro transcription is carried out according to the operation manual of the T7 High Yield RNA Synthesis Kit (Yeasen Biotechnology: 10623ES50). After the RNA products were purified, they were dissolved in 20 μL RNase-free H₂O, and the RNA yield in each reaction tube was tested using Nanodrop One.

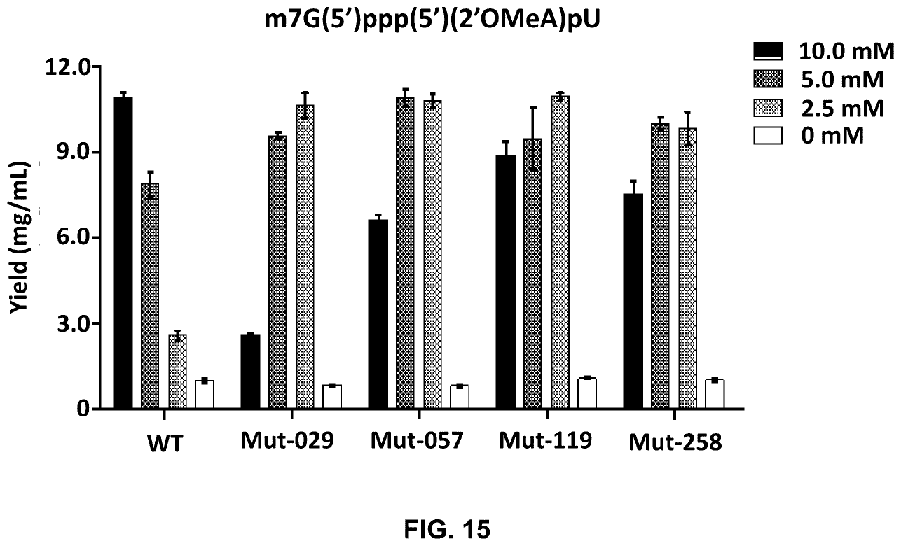

As shown in FIG. 15, in the aforementioned reaction buffer, the RNA yields of mutants Mut-029, Mut-057, Mut-119, and Mut-258 in the reaction system without the addition of cap analogs were comparable to that of the wild type T7 RNA polymerase, indicating that these mutants retained the selectivity of wild type T7 RNA polymerase for cap analogs.

At the recommended cap analog concentration (10 mM), the mutants exhibited reduced yields compared to the wild type T7 RNA polymerase, with Mut-029 yielding only 2.6 mg/mL. However, as the cap analog concentration decreased gradually, the transcription yields of the mutants gradually increased. Among them, mutants Mut-029, Mut-057, and Mut-119 reached the yield level of the wild type at 10 mM cap analog concentration when the cap analog concentration was reduced to 2.5 mM. The aforementioned results indicated that in this reaction buffer, mutants Mut-029, Mut-057, and Mut-119 were more suitable for low concentrations of cap analogs, allowing for a significant reduction in the amount of cap analogs used and thereby saving costs for in vitro transcription.

1. A mutant T7 RNA polymerase protein selected from the group of:
a1: a protein comprising a substitution at one or at least two of the amino acid residues of a wild-type T7 RNA polymerase comprising the amino acid sequence set forth in SEQ ID NO:1, the amino acid residues being selected from the group of:
I6, I19, K60, N67, A70, N86, F162, V186, N233, A247, P277, A465, L651, or N764; or
a2: a protein comprising an amino acid sequence having at least 90% sequence identity to a mutant T7 RNA polymerase protein of a1, and whose activity and enzymatic properties are substantially the same as one or more of a mutant T7 RNA polymerase protein of a1.

2. The mutant T7 RNA polymerase protein according to claim 1, wherein the protein further comprises a substitution at one or at least two of the amino acid residues selected from the group of F11, I82, D87, K180, V214, M369, Y457, H523, K610, A615, S686, or K740.

3. The mutant T7 RNA polymerase protein according to claim 1, wherein the substituted protein further comprises one or more of an additional substitution, deletion, or addition of a mutant T7 RNA polymerase protein of claim 1, the protein having substantially the same activity and enzymatic properties as one or more of a mutant T7 RNA polymerase protein of claim 1.

4. The mutant T7 RNA polymerase protein according to claim 2, wherein the protein comprises one or a combination of at least two of an amino acid substitution selected from the group of: I6G, F11L, I19T, K60I, N67S, A70Q, A70T, I82V, N86D, D87G, F162S, K180E, K180D, V186I, V214A, N233D, A247T, A247I, P277L, M369S, M369T, Y457H, A465T, H523R, K610R, A615T, L651M, S686G, K740R, or N764D.

5. The mutant T7 RNA polymerase protein according to claim 2, wherein the protein comprises one or a combination of at least two of an amino acid substitution selected from the group of: I6G, F11L, I19T, K60I, N67S, A70Q, N86D, F162S, K180E, K180D, V186I, V214A, N233D, A247I, P277L, M369S, Y457H, A465T, H523R, L651M, or N764D.

6. A mutant T7 RNA polymerase protein selected from the group of:
b1: a protein comprising a substitution at one or at least two of the amino acid residues of a wild-type T7 RNA polymerase comprising the amino acid sequence set forth in SEQ ID NO:1, the amino acid residues being selected from the group of:
I6G, F11L, I19T, K60I, N67S, A70Q, A70T, N86D, F162S, K180E, K180D, V186I, V214A, N233D, A247T, A247I, P277L, M369S, Y457H, A465T, H523R, L651M, or N764D;
b2: a protein comprising at least two of the amino acid residues of a wild-type T7 RNA polymerase comprising the amino acid sequence set forth in SEQ ID NO:1, the amino acid residues being selected from the group of:
I82V, D87G, M369T, K610R, A615T, S686G, or K740R;
b3: the protein of b1 further comprising a substitution at one or at least two of the amino acid residues of a wild-type T7 RNA polymerase comprising the amino acid sequence set forth in SEQ ID NO:1, the amino acid residues being selected from the group of:
I82V, D87G, M369T, K610R, A615T, S686G, or K740R; or
b4: a protein comprising an amino acid sequence having at least 90% sequence identity to a mutant T7 RNA polymerase protein of b1, b2, or b3, and whose enzymatic activity and properties are substantially the same as one or more of a mutant T7 RNA polymerase protein of b1, b2, or b3.

7. A mutant T7 RNA polymerase protein selected from the group of:
c1: a protein comprising a substitution at one or at least two of the amino acid residues of a wild-type T7 RNA polymerase comprising the amino acid sequence set forth in SEQ ID NO:1, wherein the substitution is selected from the group of:
(1) N86D/A615T;
(2) F162S;
(3) F162S/K180E;
(4) A247I;
(5) P277L;
(6) A70Q/F162S/K180E;
(7) N233D/A465T;
(8) D87G/N233D/V186I;
(9) A70T/P277L;
(10) N67S/I82V/Y457H/K610R/L651M/N764D;
(11) F162S/A247T;
(12) F162S/N233D/A247T;
(13) V214A;
(14) I19T/K180D/M369T;
(15) F162S/K180E/A247I;
(16) A70Q/V214A;
(17) A465T;
(18) F11L;
(19) K180E;
(20) M369S/S686G;
(21) K60I/A465T/K740R;
(22) H523R;
(23) A70T/K180D;
(24) I6G;
(25) A70Q/K180E;
(26) A70Q;
(27) V214A/A465T; or
(28) A70Q/A247T; or
c2: a protein comprising an amino acid sequence having at least 90% sequence identity to a mutant T7 RNA polymerase protein of c1, and whose activity and enzymatic properties are substantially the same as one or more of a mutant T7 RNA polymerase protein of c1.

8. The mutant T7 RNA polymerase protein according to claim 7, wherein the substituted protein further comprises one or more of an additional substitution, deletion, or addition of a mutant T7 RNA polymerase protein of claim 7, the protein having substantially the same activity and enzymatic properties as one or more of a mutant T7 RNA polymerase protein of claim 7.

9. The mutant T7 RNA polymerase protein according to claim 1, wherein the protein further comprises one or more of a tag or an enzyme cleavage site at one or both of the N-terminus or the C-terminus of the protein, and wherein the tag or the enzyme cleavage site does not substantially affect the activity and enzymatic properties of the mutant T7 RNA polymerase protein.

10.-11. (canceled)

12. The mutant T7 RNA polymerase protein according to claim 1, wherein the protein, as compared to a wild-type T7 RNA polymerase comprising the amino acid sequence set forth in SEQ ID NO:1, is characterized by producing during transcription one or more of a lower amount of dsRNA by-products, a higher product integrity, a higher yield, a higher transcriptional activity, or a lower cap analog usage.

13. The mutant T7 RNA polymerase protein according to claim 1, wherein the protein is characterized by being capable of catalyzing co-transcriptional capping.

14. A composition comprising the mutant T7 RNA polymerase protein according to claim 1, and one or more of an in vivo transcription reagent or in vitro transcription reagent.

15. A transcription kit comprising a mutant T7 RNA polymerase protein according to claim 1.

16. The transcription kit according to claim 15, wherein the transcription kit is a co-transcription capping kit.

17. The transcription kit according to claim 16, further comprising a cap analog and a buffer system.

18. (canceled)

19. A method of generating a transcription product of a deoxyribonucleic acid (DNA) comprising contacting the DNA with a mutant T7 RNA polymerase protein according to claim 1.

20. The method according to claim 19, further comprising contacting the transcription product with a capping enzyme to form a capped transcription product.

21. The method according to claim 19, further comprising combining the transcription product or the capped transcription product with a pharmaceutically acceptable additive to produce a pharmaceutical dosage form.

22. A method of generating a capped transcription product of a deoxyribonucleic acid (DNA) comprising:
(i) mixing the DNA, a cap analog, and a mutant T7 RNA polymerase protein according to claim 1, in a buffer system; and
(ii) generating the capped transcription product through transcription of the DNA using the mixture of step (i).

23. The method of claim 22, further comprising combining the capped transcription product with a pharmaceutically acceptable additive to produce a pharmaceutical dosage form.
